SEQUENCE LISTING (16 sequences)

SEQ ID NO: 1 - DNA, 5361 bp, Homo sapiens
  source 1..5361: mol_type = unassigned DNA
tttctccttccctccacgggccgggtgagaaagtagccgggggctatcccgacccggcggttcttggggagggggccgaacaagaaaagggaggagatggagataacttccccggatttagcttttttgtctttgtttttgttctcaccacttccatcggatgactggagagtaaaagggaacccggagcggggtggcgagcagcgctttgagaaaatgcaggagtgtgtttggagacgcgtaaagttgcctttcaagctctggcctccgggcacgcgatgctccgcggcgggctgactcagggctgccttgggcctccctgccaccctcctggaaatgatgcaagtcctgactgtcacctggatccctgcagcccagcctggaatgcgtctggattaggggaaagacgagaaacgacactccaggtgttgcacggcccaccaaagcgggaagatagggcagttgctcagaccaaatactgtatctagtgcttctgctcctatcttcaatcgtggggttctttttaatgcaaagtgtcacaaggccaggaattcccatgtgtgctcagttggcccacagcatcattgtgcctaggaaactgcttcaatttatcaagtcctctgggctgggaatctcactgaattccaaacggcggaaagaggaaactttcccaacccgatgtgggtgtgacgcgagccaggggccccagggacactgtcccagagcacaccgtccccctttaacagcaactggagcttggattcgctcttatattgtacagtcctttcgaccattgccctggagcacccgcacacgcgcacgcatctccggccgcgctcacacacactcatacacacgcacgcaaacgcgtggccgccgccaggtcggcaactttgtccggcgctcccagcggcgctcggcttcctcctgtagtagttgagcgcaggccccgcctcccggccgtgttgtcaaaagggccggggtctcggattggtccagccgccgggacaacacctgctcgactccttcattcaagtgacaccagagcttccagggatatttgaggcaccatccctgccattgccgggcactcgcggcgctgctaacggcctggtcacatgctctccggagagctacgggagggcgctgggtaacctctatccgagccgcggccgcgaggaggagggaaaaggcgagcaaaaaggaagagtgggaggaggaggggaagcggcgaaggaggaagaggaggaggaggaagaggggagcacaaaggatccaggtctcccgacgggaggttaataccaagaaccatgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtgaaatgaaaggcctggccacgaaatctttcctccacgccgtccattttcttatctatggacattccaaaacatttaccattagagaggggggatgtcacacgcaggattctgtggggactgtggacttcatcgaggtgtgtgttcgcggaacggacaggtgagatggagacccctggggccgtggggtctcaggggtgcctggtgaattctgcacttacacgtactcaagggagcgcgcccgcgttatcctcgtacctttgtcttctttccatctgtggagtcagtgggtgtcggccgctctgttgtgggggaggtgaaccagggaggggcagggcaaggcagggcccccagagctgggccacacagtgggtgctgggcctcgccccgaagcttctggtgcagcagcctctggtgctgtctccgcggaagtcagggcggctggattccaggacaggagtgaatgtaaaaataaatatcgcttagaatgcaggagaagggtggagaggaggcaggggccgagggggtgcttggtgccaaactgaaattcagtttcttgtgtggggccttgcggttcagagctcttggcgagggtggagggaggagtgtcatttctatgtgtaatttctgagccattgtactgtctgggctgggggggacactgtccaagggagtggcccctatgagtttatattttaaccactgcttcaaatctcgatttcactttttttatttatccagttatatctacatatctgtcatctaaataaatggctttcaaacaaagcaactgggtcattaaaaccagctcaaagggggtttaaaaaaaaaaaaccagcccatcctttgaggctgatttttcttttttttaagttctattttaaaagctatcaaacagcgacatagccatacatctgactgcctgacatggactcctgcccacttgggggaaaccttatacccagaggaaaatacacacctggggagtacatttgacaaatttcccttaggatttcgttatctcaccttgaccctcagccaagattggtaaagctgcgtcctggcgattccaggagacccagctggaaacctggcttctccatgtgaggggatgggaaaggaaagaagagaatgaagactacttagtaattcccatcaggaaatgctgaccttttacataaaatcaaggagactgctgaaaatctctaagggacaggattttccagatcctaattggaaatttagcaataaggagaggagtccaaggggacaaataaaggcagagagaagagacagaactaaaaatacgaggaaaggagagtgaggattttcattaaaagtctcagcagtgggtttcttgggttatttaaaacatcacctaaataggccttttcttcctaattggccatcaaattaaagcctatcctttctcaagcaggagctggtattgtagggagtggccgggtattctgggctgggctcttctggagtagggggtcagcaaacattgtctgcaaagggccagatactgaatccagtactttcagtttggcgagccgtgaggtctctgtcgaaactactcaactctgccgtcctagcacaaaagcagccatagacaacacacaaacgagagggcttggctcccttccaggaagatttatttaacaggctcccagctgaatttcactcacaggacacagtttactgatctctgttctagtgagtgggtcaaaaagcatatgcatccttatccgtcaactcatcagctcttcctcaaggcaacctgaggccagacaccaagaaaccaagcgtatctgctctaaaatgacttgttcctggggaatgccttcaaccaaaacacagctagtatttctatgccccaaatccaatcccagtctttcatgatccatgccggcggttgggtggggaggggaatcattggttgggggaagggaggaaaccccacctccagcccccgccaccgggctccctgggcacccagcaagatctggggctgcagagaacagaagagctggtgcacttaatccagctctgcccttggggggaggaggacctgtgtgtcaggctctgccatgggaacgagtgtaaaccgtggctgtctcctgcagtgagccaccgcggcaggcacgttgactgttttactgacatcactcaaaagctaaagcaataacattctcctgcgttgctgagtcagctgttcatttgtccgccagctcctggactggatgtgtgaaaggcatcacatttccattttcctccgtgtaaatgttttatgtgttcgcctactgatcccattcgttgcttctattgtaaatatttgtcatttgtatttattatctctgtgttttccccctaaggcataaaatggtttactgtgttcatttgaacccatttactgatctctgttgtatatttttcatgccactgctttgttttctcctcagaagtcgggtagatagcatttctatcccatccctcacgttattggaagcatgcaacagtatttattgctcagggtcttctgcttaaaactgaggaaggtccacattcctgcaagcattgattgagacatttgcacaatctaaaatgtaagcaaagtagtcattaaaaatacaccctctacttgggctttatactgcatacaaatttactcatgagccttcctttgaggaaggatgtggatctccaaataaagatttagtgtttattttgagctctgcatcttaacaagatgatctgaacacctctcctttgtatcaataaatagccctgttattctgaagtgagaggaccaagtatagtaaaatgctgacatctaaaactaaataaatagaaaacaccaggccagaactatagtcatactcacacaaagggagaaatttaaactcgaaccaagcaaaaggcttcacggaaatagcatggaaaaacaatgcttccagtggccacttcctaaggaggaacaaccccgtctgatctcagaattggcaccacgtgagcttgctaagtgataatatctgtttctactacggatttaggcaacaggacctgtacattgtcacattgcattatttttcttcaagcgttaataaaagttttaaataaatggcaaaaaaaaaaaaaaaaaa

SEQ ID NO: 2 - DNA, 909 bp, Homo sapiens
  source 1..909: mol_type = unassigned DNA
atgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtga

SEQ ID NO: 3 - protein, 302 aa, Homo sapiens
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQKPPTAPPERQPQVDRTKLSRAHHGEAGHHLPEPSSRETGRGAKGERGSKSHPNAHARGRVGGLGAQGPSGSSEWEDEQSEYSDIRR

SEQ ID NO: 4 - protein, 302 aa, Canis lupus
MCAERLGQFVTLALVLATFDLARGTDATNPPEGPQDRGSQQKGRLSLQNTAEIQHCLVNAGDVGCGVFECFENNSCEIRGLHGICMTFLHNAGKFDAQGKSFIKDALKCKAHALRHRFGCISRKCPAIKEMVFQLQRECYLKHDLCSAAQENTRVMVEMIHFKDLLLHEPYVDLVNLLLTCGEEVKEAITHSVQAQCEQNWGSLCSILSFCTSAIQRPPTVPPERQLQVDRAKLSKTHHGEAGHHVSEPSSWETGRGTKGEGGSKSHLNAHARGRAVGQGGQGTSGSSEWEEEPSEYSDIRR

SEQ ID NO: 5 - protein, 302 aa, Felis catus
MCAERLGQFVTLALVLATFDPARGTDATNPPEGPQDRGSQQKGRLSLQNTAEIQHCLVNAGDVGCGVFECFENNSCEIRGLHGICMTFLHNAGKFDAQGKSFIKEALKCKAHALRHRFGCISRKCPAIKEMVFQLQRECYLKHDLCSAAQENTRVMVEMIHFKDLLLHEPYVDLVNLLLTCGEEVKEAITHSVQAQCEQNWGSLCSILSFCTSAIQRPPTVPPERQLQVDRAKLSRAHHGDTGHHLSEASSRETGRGVKGERGSKSHPNAHARGRAAGHGGQGTSGSSEWEEEQSEYSDIRR

SEQ ID NO: 6 - protein, 302 aa, Equus caballus
MCTERLGQFMTLALVLATFDPARGTDATHPPEGPQDRGSQQKGRLSLQNTAEIQHCLVNAGDVGCGVFECFENNSCEIRGLHGICMTFLHNAGKFDAQGKSFIKDALKCKAHALRHRFGCISRKCPAIKEMVFQLQRECYLKHDLCSAAQENTRVMVEIIHFKDLLLHEPYVDLVNLLLTCGEEVKEAITHSVQAQCEQSWGSLCSILSFCTSAIQRPPTVPPERRLQGDRARLSRAHHEEAGHHLSEPSSQETGRGAKGERGSKSHPNTHARGRAGGHGAQGTSGSSEWEDEQSEYSDIRR

SEQ ID NO: 7 - protein, 302 aa, Bos taurus
MCAERLGQFVTLALVLVTFDPARGTDATNTPEGPQDRGSQQKGRLSLQNTAEIQHCLVNAGDVGCGVFECFENNSCEIRGLHGICMTFLHNAGKFDAQGKSFIKDALKCKAHALRHRFSCISRKCPAIKEMVFQLQRECYLKHDLCSAAQENIRVMVEMIHFKDLLLHEPYVDLVNLLLTCGEEVKEAITHSVQAQCEQSWGSLCSILSFCTSAIQRPPTAPPEHQLQGDRAKLSRGHPAEMGHHLAEPSSRETGRGAKGERGSKSHPNAHARGRAAGPGAQGTSGSSEWEDEQSEYSDIRR

SEQ ID NO: 8 - protein, 301 aa, Sus scrofa
MCAERLGQFVTLALLLATFDPARGTEATNPPESPQDRGSQQKGRLSLQNTAEIQHCLVNAGDVGCGVFECFENNSCEIRGLHGICMTFLHNAGKFDAQGKSFIKDALKCKAHALRHRFGCISRKCPAIKEMVFQLQRECYLKHDLCSAAQENTRVMVEMIHFKDLLLHEPYVDLVNLLLTCGEEVKEAITHSVQAQCEQNWGSLCSILSFCTSAIQRPPTAPPEHPPQGDRAKPSRAHGEAGHPLSEPSSRETGRGAKGERASKSHPIAHARGRAAGHGAQGTSGSSEWEDEQSEYSDIRR

SEQ ID NO: 9 - protein, 302 aa, Oryctolagus cuniculus
MCAERLGRFVTLTLVFATFDLARGTDATHPPEGLPDRGSQQKGRLSLQNTAEIQHCLVNAGDVGCGVFECFENNSCEIRGLHGICMTFLHNAGKFDAQGKSFIKDALKCKAHALRHRFGCISRKCPAIKEMVFQLQRECYLKHDLCSAAQDNIRVIVEMIHFKDLLLHEPYVDLVNLLLTCGEEVKEAVTHSVQAQCEQNWGGLCSILSFCTPTIQRPPTALPERQLQVDRTKLPRAHHGEAGPHHAEPSSKETSRGAKGERGSRSHPNAHARGRAGSPGAQGPSGSSEWEDEQSEYSDIRR

SEQ ID NO: 10 - protein, 302 aa, Ovis aries
MCAERLGQFVTLALVLVTFDPARGTDATNTPEGPQDRGSQQKGRLSLQNTAEIQHCLVNAGDVGCGVFECFENNSCEIRGLHGICMTFLHNAGKFDAQGKSFIKDALKCKAHALRHRFSCISRKCPAIKEMVFQLQRECYLKHDLCSAAQENIRVMVEMIHFKDLLLHDPTVDLVNLLLTCGEEVKEAITHSVQAQCEQSWGSLCSILSFCTSAIQRPPTAPPERQLQGDRAKLSRGHPAEMGHHLAEPSSRETGRGAKGERGSKSHPNAHARGRTAGPGAQGTSGSSEWEDEQSEYSDIRR

SEQ ID NO: 11 - protein, 302 aa, Macaca nemestrina
MCAERLGHFMTLALVLATIDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVFQLQRECYLKHDLCAAAQENTRVIVEMIHFKDLLLHEPYVDLVNLLLTCGEEVKEAITHSVQVQCEQNWGSLCSILSFCTSAIQRPPTAPPERQPQVDRAKLSRAHHGEAGHHLPEPSSRETGRGAKGERGSKSHPNAHARGRVGGLGAQGPSGSSEWEDEQSEYSDIRR

SEQ ID NO: 12 - protein, 302 aa, Pan troglodytes
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQRPPTAPPERQPQVDRTKLSRAHHGEAGHHLPEPSSRETGRGAKGERGSKSHPNAHARGRVGGLGAQGPSGSSEWEDEQSEYSDIRR

SEQ ID NO: 13 - protein, 296 aa, Mus musculus
MCAERLGQFVTLALVFATLDPAQGTDSTNPPEGPQDRSSQQKGRLSLQNTAEIQHCLVNAGDVGCGVFECFENNSCEIQGLHGICMTFLHNAGKFDAQGKSFIKDALRCKAHALRHKFGCISRKCPAIREMVFQLQRECYLKHDLCSAAQENVGVIVEMIHFKDLLLHEPYVDLVNLLLTCGEDVKEAVTRSVQAQCEQSWGGLCSILSFCTSNIQRPPTAAPEHQPLADRAQLSRPHHRDTDHHLTANRGAKGERGSKSHPNAHARGRTGGQSAQGPSGSSEWEDEQSEYSDIRR

SEQ ID NO: 14 - protein, 296 aa, Rattus norvegicus
MCAERLGQFVTLALVFATLDPARGTDSTNPPEGPQDRGSQQKGRLSLQNTAEIQHCLVNAGDVGCGVFECFENNSCEIQGLHGICMTFLHNAGKFDAQGKSFIKDALRCKAHALRHKFGCISRKCPAIREMVYQLQRECYLKHDLCSAAQENVVVIVEMIHFKDLLLHEPYVDLVNLLLTCGEDVREAVTRSVQAQCEQSWGGLCSILSFCTSNIQRPPTAAPEHQPLADRAQLSRPYHRDTDHHLTANRGAKGERGSKSHLHAHARGGAGGQSAQGPSGSSEWEDEQSEYSDIRR

SEQ ID NO: 15 - protein, 302 aa, Cavia porcellus
MCAERLGQFVTLALVLATFDPARGTDATNPPEGPQDRGSQQKGRLSLQNTADIQHCLVNAGDVGCGVFECFENNSCEIRGLHGICMTFLHNAGKFDAQGKSFIKDALKCKAHALRHRFGCISRKCPAIKEMVFQLQRECYLKHDLCAAAQENTRVLVEMIHFKDLLLHEPYVDLVNLLLTCGEEVKEAISHSVQAQCEQNWGGLCSILSFCTSAMHRPPTVSPEHQLQMDRARLSRTHQGEASHYFSEPSSRESIRDAKGERGSKGHSNAHARGRAGGHGAQGPSGSSEWEDEQSEYSDIRR

SEQ ID NO: 16 - protein, 30 aa, synthetic construct
  REGION 1..30: note = Description of Artificial Sequence: Synthetic polypeptide
  MOD_RES 6: note = Glycosylated Ser
  MOD_RES 12: note = Glycosylated Ser
  MOD_RES 17: note = Glycosylated Ser
  MOD_RES 23: note = Glycosylated Ser
FQSSSSKAPPPSLPSPSRLPGPSDTPILPQ

(19) United States
(12) Patent Application Publication
(10) Pub. No.: US 2024/0033327 A1
(43) Pub. Date: Feb. 1, 2024

(54) LNP AND LIPOSOME COMPOSITIONS FOR LONGEVITY IN MAMMALS AND METHODS OF USING THE SAME

(71) Applicant: Cellular Longevity, Inc., San Francisco, CA (US)

(72) Inventors: Celine-Lea HALIOUA-HAUBOLD, San Francisco, CA (US); Michael LACROIX-FRALISH, Putnam Valley, NY (US); Dina JUAREZ-SALINAS, San Francisco, CA (US)

(21) Appl. No.: 18/201,901

(22) Filed: May 25, 2023

Related U.S. Application Data
(63) Continuation of application No. PCT/US2021/061092, filed on Nov. 30, 2021.
(60) Provisional application No. 63/119,546, filed on Nov. 30, 2020.

(51) Int. Cl. A61K 38/22; A61K 9/51; A61K 9/127; A61K 48/00; A61P 39/00

(52) U.S. Cl. CPC A61K 38/22; A61K 9/5123; A61K 9/127; A61K 48/0033; A61P 39/00

(57) ABSTRACT

Disclosed herein are compositions and methods for increasing the lifespan of a non-rodent mammal or a mature mammal in need thereof. The compositions and methods comprise therapeutically effective amounts of a nucleic acid encoding a target protein or a functional fragment or variant thereof or therapeutically effective amounts of a modified protein comprising target protein or a functional fragment or variant thereof.

### CROSS-REFERENCE

This application is a continuation of International Application No. PCDUS2,021/061092, filed Nov. 30, 2021 which claims the benefit of U.S. Provisional Application No. 63/119,546, filed Nov. 30, 2020, which is entirely incorporated herein by reference.

### SEQUENCE LISTING

The instant application contains a Sequence Listing which has been submitted electronically in XML format and is hereby incorporated by reference in its entirety. Said XML copy, created on Jun. 14, 2023, is Named 58989-703_301_SL.Xml and is 26,878 Bytes in Size

### BACKGROUND

Chronological age is well understood to be the single greatest risk factor for nearly every major cause of mortality and morbidity in living organisms, including humans and companion dogs. Even before the development of observable disease, the physiology of organ systems and tissues progressively declines throughout life. Throughout history, products and methods that promote longevity and extend lifespan have been eagerly sought. Generally, these products and methods have proven to be ineffective and/or unsafe. Accordingly, there remains an unmet need for safe and effective products and methods that promote longevity and extend lifespan.

### SUMMARY

In one aspect, provided herein is a method for increasing lifespan in a non-rodent mammal in need thereof comprising: administering to the mammal a therapeutically effective amount of a composition comprising a lipid nanoparticle or liposome, wherein the lipid nanoparticle or liposome comprises a nucleic acid encoding a target protein or a functional fragment or variant thereof.

In another aspect, provided herein is a method for increasing lifespan in a mature mammal in need thereof comprising: administering to the mammal a therapeutically effective amount of a composition comprising a lipid nanoparticle or liposome, wherein the lipid nanoparticle or liposome comprises a nucleic acid encoding a target protein or a functional fragment or variant thereof.

In some embodiments, the lipid nanoparticle or liposome comprises at least one of the following: (a) a cationic lipid; (b) a phospholipid; (c) a cholesterol or a derivative thereof; and/or (d) a conjugated lipid that inhibits aggregation of particles.

In some embodiments, the nucleic acid comprises RNA. In some specific embodiments, the RNA is an mRNA comprising one or more of 5′ cap structure, a Kozak consensus sequence, a 5′-UTR, a 3′-UTR, and a 3′ poly(A) tail.

In other embodiments, the nucleic acid comprises DNA that is single stranded or double stranded. In some specific embodiments, the DNA is linear DNA or circular DNA. In some embodiments, the DNA comprises a promoter. In some specific embodiments, the DNA comprises a CMV, CAG, CBA, EF1a, RSV, MSCV, ALB, or TBG promoter sequence. In some embodiments, the DNA comprises a termination and/or polyadenylation sequence. In some specific embodiments, the DNA comprises an SV40, hGH, BGH, or rbGlob termination and/or polyadenylation sequence.

In some embodiments, the administering is repeated at least one additional time. In other embodiments, the administering is not repeated or is not repeated for at least a week, at least a month, at least 6 months, at least a year, at least two years, or at least five years after initial administering.

In some embodiments, the nucleic acid further encodes a modification that improves the half-life of the target protein or the functional fragment or variant thereof in serum. In some embodiments, the nucleic acid further encodes a linker located between the target protein or the functional fragment or variant thereof and the modification that improves the half-life. In some specific embodiments, the linker comprises between about 2 amino acids and about 500 amino acids.

In some embodiments, administering the composition comprises intravenous injection or infusion, intraperitoneal injection, intramuscular injection, or subcutaneous injection.

In some embodiments, the nucleic acid encodes a fragment of stanniocalcin-2 (STC-2). In some embodiments, the nucleic acid encodes stanniocalcin-2 (STC-2).

In some embodiments, the nucleic acid encodes a peptide growth hormone receptor antagonist or a fragment thereof. In some embodiments, the nucleic acid encodes a growth hormone releasing hormone antagonists or a fragment thereof. In some embodiments, the nucleic acid encodes a peptide inhibitor of PAPP-A or a fragment thereof.

In another aspect, provided herein is a method for increasing lifespan in a non-rodent mammal in need thereof comprising: administering to the mammal a therapeutically effective amount of a composition including a modified protein comprising a target protein or a functional fragment or variant thereof and a modification that improves the half-life of the target protein or the functional fragment or variant thereof in serum.

In another aspect, provided herein is a method for increasing lifespan in a mature mammal in need thereof comprising: administering to the mammal a therapeutically effective amount of a composition including a modified protein comprising a target protein or a functional fragment or variant thereof and a modification that improves the half-life of the target protein or the functional fragment or variant thereof in serum.

In some embodiments, the modified protein is incapable of crossing the blood brain barrier.

In some embodiments, administering the composition comprises a systemic administration selected from intravenous injection and intravenous infusion.

In some embodiments, the modification that improves the half-life of the modified protein in serum is derived from an Fc domain of an antibody, a transferrin, an albumin, a CTP sequence, an XTEN sequence, an ELP sequence, a PAS sequence, a HAP sequence, and a GLK sequences, or a combination thereof. In some specific embodiments, the antibody is IgG (e.g., IgG1, IgG2, IgG3, and IgG4), IgA (e.g., IgA1 and IgA2), IgD, and IgE. In some specific embodiments, the Fc domain is from an IgG4 and comprises one or more mutations selected from S228P, T250Q, M428L, V308T, L309P, and Q311S in accordance with Kabat numbering.

In some embodiments, the modified protein further comprises a linker located between the target protein or the functional fragment or variant thereof and the modification that improves the half-life in serum. In some embodiments, the linker is a polypeptide linker comprising between about 2 amino acids and about 500 amino acids, or is a synthetic linker, e.g., comprising PEG.

In some embodiments, the composition further comprises a lipid nanoparticle or liposome.

In some embodiments, the administering is repeated at least one additional time, In other embodiments, the administering is not repeated or not repeated or is not repeated for at least a week, at least a month, at least 6 months, at least a year, at least two years, or at least five years after initial administering.

In some embodiments, the target protein comprises or is a fragment of stanniocalcin-2 (STC-2). In some specific embodiments, the fragment of STC-2 is capable of binding pregnancy-associated plasma protein-A (PAPP-A) and/or PAPP-A2 and inhibiting their enzymatic activity.

In some embodiments, the target protein is a peptide growth hormone receptor antagonist or a fragment thereof. In some embodiments, the target protein is a growth hormone releasing hormone antagonists or a fragment thereof. In some embodiments, the target protein is a peptide inhibitor of PAPP-A or a fragment thereof.

In some embodiments, the mammal has not reached maturity.

In some embodiments, the mammal is dog, cat, horse, cow, pig, rabbit, sheep, or non-human primate, e.g., the mammal is a dog. In other embodiments, the mammal is a human. In other embodiments, the mammal is a rodent, e.g., a mouse and a rat.

In some embodiments, increasing lifespan comprises an increase of at least 5% in lifespan relative to the expected or median lifespan of a mammal of similar species, strain, or breed. In other embodiments, increasing lifespan comprises an increase of at least 10%, at least 15%, at least 20%, or at least 25% in lifespan.

In some embodiments, the method provided herein further comprises detecting an amount of one or more biomarkers in a first blood sample. In some embodiments, the first blood sample is obtained from the mammal before administering the composition. In other embodiments, the method provided herein further comprises detecting an amount of one or more biomarkers or the one or more biomarkers in a second blood sample. In some embodiments, the second blood sample is obtained from the mammal after administering the composition.

In some embodiments, the therapeutically effective amount of the composition produces an increase in the amount of one or more biomarkers selected from phosphorylated IGF-1R, glucose, insulin, non-esterified fatty acids (NEFA), cholesterol, thyroid hormones, GH-receptor, phosphorylated GH-receptor, insulin, IGFBP1, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, Rac, mechanistic target of rapamycin (mTOR), AMP-activated protein kinase (AMPK), IL-6, TNFα-receptor I or II, IL-8, IL-2, interferon gamma, adiponectin, FGF21, FGF23, GDF15, Cystatin C, NT-proBNP, FSTL3, total homocysteine (tHcy), alpha Klotho, beta Klotho, Glucose transporter type 4 (GLUT4), serum triglycerides, serum free fatty acids, Dehydroepiandrosterone sulfate (DHEAS), NT-proBNP, Follistatin-like 3 FSTL3, myostatin (GDF8), Transforming growth factor beta (TGF-β) or a homolog thereof in the second sample relative to the first sample and/or relative to a historical control, and wherein optionally the therapeutically effective amount of the composition produces an increase in insulin sensitivity. In other embodiments, the therapeutically effective amount of the composition produces a decrease in the amount of one or more biomarkers selected from phosphorylated IGF-1R, glucose, insulin, non-esterified fatty acids (NEFA), cholesterol, thyroid hormones, GH-receptor, phosphorylated GH-receptor, insulin, IGFBP1, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, Rac, mechanistic target of rapamycin (mTOR), AMP-activated protein kinase (AMPK), IL-6, TNFα-receptor I or II, IL-8, IL-2, interferon gamma, adiponectin, FGF21, FGF23, GDF15, Cystatin C, NT-proBNP, FSTL3, total homocysteine (tHcy), alpha Klotho, beta Klotho, Glucose transporter type 4 (GLUT4), serum triglycerides, serum free fatty acids, Dehydroepiandrosterone sulfate (DHEAS), NT-proBNP, Follistatin-like 3 FSTL3, myostatin (GDF8), Transforming growth factor beta (TGF-β) or a homolog thereof in the second relative to the first sample and/or relative to a historical control. In other embodiments, the therapeutically effective amount of the composition produces a change in the amount of one or more biomarkers selected from phosphorylated IGF-1R, glucose, insulin, non-esterified fatty acids (NEFA), cholesterol, thyroid hormones, GH-receptor, phosphorylated GH-receptor, insulin, IGFBP1, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, Rac, mechanistic target of rapamycin (mTOR), AMP-activated protein kinase (AMPK), IL-6, TNFα-receptor I or II, IL-8, IL-2, interferon gamma, adiponectin, FGF21, FGF23, GDF15, Cystatin C, NT-proBNP, FSTL3, total homocysteine (tHcy), alpha Klotho, beta Klotho, Glucose transporter type 4 (GLUT4), serum triglycerides, serum free fatty acids, Dehydroepiandrosterone sulfate (DHEAS), NT-proBNP, Follistatin-like 3 FSTL3, myostatin (GDF8), Transforming growth factor beta (TGF-β) or a homolog thereof in the second sample relative to the first sample and/or relative to a historical control.

In some embodiments, the therapeutically effective amount of the composition produces a change in an epigenetic marker in the second sample relative to the first sample and/or relative to a historical control. In some specific embodiments, the epigenetic marker is an amount of methylated DNA. In specific cases, the methylated DNA is characterized via one or more of the Horvath epigenetic method, PhenoAge method, Hannum epigenetic method, or GrimAge method. In some embodiments, the epigenetic marker is one or more of DNA sequences covalently modified by cytosine methylation and hydroxymethylation, or of histone proteins by lysine acetylation, lysine and arginine methylation, serine and threonine phosphorylation, lysine ubiquitination, or sumoylation.

In some embodiments, the target protein is a fragment of STC-2, and the therapeutically effective amount of the composition produces a decrease in the amount of free IGF-1 to IGF-1 that is bound to an insulin-like growth factor-binding protein (IGFBP, e.g., one of IGFBP1 to IGFBP6) in the second sample relative to the first sample and/or relative to a historical control.

In some embodiments, the target protein is a fragment of STC-2, and the therapeutically effective amount of the composition produces a decrease in the amount of cleaved insulin-like growth factor-binding protein (IGFBP, e.g., one of IGFBP1 to IGFBP6) in the second sample relative to the first sample and/or relative to a historical control. In some specific embodiments, the IGFBP is IGFBP-4 or is IGFBP-5.

In some embodiments, the target protein is a fragment of STC-2, and the therapeutically effective amount of the composition produces a decrease in the enzymatic activity of pregnancy-associated plasma protein A (PAPP-A) and/or PAPP-A2 in the second sample relative to the first sample and/or relative to a historical control.

In some embodiments, administering the composition further improves the healthspan of the mammal. In some specific embodiments, improving the healthspan of the mammal comprises treating a cancer and/or delaying or preventing the onset of the cancer in the mammal. In some specific embodiments, the therapeutically-effective amount of a composition is capable of treating a cancer and/or preventing or delaying the onset of the cancer in the mammal.

In some embodiments, the composition is a lipid nanoparticle or liposome, and the nucleic acid is encapsulated in the lipid nanoparticle.

In some embodiments, the lipid nanoparticle or liposome comprises one or more of the following: (a) a cationic lipid; (b) a phospholipid; (c) a cholesterol or a derivative thereof; and/or (d) a conjugated lipid that inhibits aggregation of particles. In some specific embodiments, the cationic lipid comprises 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoley oxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoley loxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido) ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis, cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or a combination thereof. In specific cases, the cationic lipid comprises 10 mol % to 70 mol % of total lipid present in the lipid nanoparticle. In some specific embodiments, the phospholipid comprises dipalmitoylphosphatidylcholine (DPPC), di stearoylpho sphatidy lcholine (D SPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), or combination thereof. In specific cases, the phospholipid comprises 10 mol % to 70 mol % of total lipid present in the lipid nanoparticle.

In some specific embodiments, the cholesterol or derivative thereof comprises cholestanol, cholestanone, cholestenone, coprostanol, cholesteryl-2′-hydroxyethyl ether, cholesteryl-4 hydroxybuty 1 ether, or a combination thereof. In specific cases, the cholesterol or derivative thereof comprises 10 mol % to 70 mol % of total lipid present in the lipid nanoparticle. In some specific embodiments, the conjugated lipid comprises a PEG-diacylglycerol (DAG), a PEG dialkyloxypropyl (DAA), a PEG-phospholipid, a PEG-ceramide (Cer), or mixtures thereof. In specific cases, the conjugated lipid that inhibit aggregation of particles comprises 10 mol % to 70 mol % of total lipid present in the lipid nanoparticle.

In some embodiments, the method provided herein further comprises a pharmaceutically acceptable carrier.

In another aspect, provided herein is a method for increasing lifespan in a non-rodent mammal in need thereof comprising: administering to the mammal a therapeutically effective amount of a composition comprising a nucleic acid encoding a target protein or a functional fragment or variant thereof, wherein the composition comprises a viral expression vector.

In another aspect, provided herein is a method for increasing lifespan in a mature mammal in need thereof comprising: administering to the mammal a therapeutically effective amount of a composition comprising a nucleic acid encoding a target protein or a functional fragment or variant thereof, wherein the composition comprises a viral expression vector.

In some embodiments, the viral expression vector is a lentivirus or an adeno-associated virus (AAV). In some specific embodiments, the AAV is AAV1, AAV2, AAV3, AAV4, AAV5, AAV6, AAV7, AAV8, AAV9, AAVrh8, AAVrh8R, AAV10, AAVrh10, AAV11, or AAV12.

In some embodiments, the nucleic acid is a DNA comprising a promoter. In some specific embodiments, the DNA comprises a CMV, CAG, CBA, EF1a, RSV, MSCV, ALB, or TBG promoter sequence. In other embodiments, the nucleic acid is a DNA comprising a termination and/or polyadenylation sequence. In some specific embodiments, the DNA comprises an SV40, hGH, BGH, or rbGlob termination and/or polyadenylation sequence.

In another aspect, provided herein is a lipid nanoparticle composition for use in a method of increasing lifespan in a non-rodent mammal, wherein the lipid nanoparticle comprises (i) a nucleic acid encoding a target protein or a functional fragment or variant thereof and (ii) one or more of the following: (a) a cationic lipid; (b) a phospholipid; (c) a cholesterol or a derivative thereof; and/or (d) a conjugated lipid that inhibits aggregation of particles. In some embodiments, the non-rodent mammal is a dog. In some embodiments, the composition comprises about 100 ng to about 2000 ng of the nucleic acid.

Yet another aspect of the present disclosure is a composition for use in any herein-described method.

It shall be understood that different aspects and/or embodiments of the disclosure can be appreciated individually, collectively, or in combination with each other. Various aspects and/or embodiments of the disclosure described herein may be applied to any of the uses set forth below and in other methods for increasing lifespan in a mammal. Any description herein concerning a specific composition and/or method apply to and may be used for any other specific composition and/or method as disclosed herein. Additionally, any composition disclosed herein is applicable to any herein-disclosed method. In other words, any aspect or embodiment described herein can be combined with any other aspect or embodiment as disclosed herein.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### DETAILED DESCRIPTION OF THE DISCLOSURE

The present disclosure is based, in part, on the discovery of compositions and methods that promote longevity and increase lifespan in a mammal. The compositions and methods further improve the healthspan of the mammal, which may include treating a cancer and/or delaying or preventing the onset of the cancer in the mammal.

### INTRODUCTION

The physiology of organ systems and tissue progressively declines through age. The accumulation of measurable cellular and physiological can cause the changes responsible for progressive increase in risk of debilitating disease and death. There are several molecular processes that have been identified to contribute to the phenomenon of biological aging with evidence linking specific mechanisms and genetic permutations with extraordinary lifespan and healthspan in humans. There is a need for developing of drugs targeting the mechanisms of cellular aging, effectively dampening multiple age-related diseases simultaneously, which would lead to tremendous value to the health care of humans and mammals.

Companion dogs are the ideal translational model for aging drug development. Compared to mice, dogs naturally develop the same age-related diseases that humans do, at approximately the same time in their lifespan. The unique genetic architecture that has arisen as a result of domestication and selective breeding of the companion dog has produced Earth's most phenotypically variable mammal, far surpassing all other domesticated animals. This provides unique opportunities for robust mechanistic comparisons and the identification of genetically-supported targets between dog breeds. Finally, unlike other species used for research, companion animals share our environments, eat varied diets, experience varied stimuli, and have access to sophisticated life-long medical care, all of which contribute to the translational validity of outcome studies. These factors combined with a lifespan that permits feasible investigation of age-related phenotypes on a reasonable time scale makes the companion dog the optimal choice for translational aging research. Traditional small molecule and protein drug approaches have been historically challenging to induce over extended period of time.

In one aspect, the methods and compositions described herein comprise a nucleic acid encoding a target protein, or a functional fragment or variant thereof. In some embodiments, the nucleic acid encoding a fragment of stanniocalcin-2 (STC-2). In some embodiments, the nucleic acid encodes a peptide growth hormone receptor antagonist or a fragment thereof. In some embodiments, the nucleic acid encodes a growth hormone releasing hormone antagonists or a fragment thereof. In some embodiments, the nucleic acid encodes a peptide inhibitor of PAPP-A or a fragment thereof.

The insulin-like growth factors (IGFs) are proteins with high sequence similarity to insulin. IGFs are part of a complex system that cells use to communicate with their physiologic environment. This complex system (often referred to as the IGF “axis”) consists of two cell-surface receptors (IGF1R and IGF2R), two ligands (Insulin-like growth factor 1 (IGF-1) and Insulin-like growth factor 2 (IGF-2)), a family of six high-affinity IGF-binding proteins (IGFBP-1 to IGFBP-6), as well as associated IGFBP degrading enzymes. Studies have shown that the Insulin/IGF axis play an important role in aging. Worms, fruit-flies, and mice have been shown to have an increased life span when homologs of genes in the IGF axis have been knocked out.

Insulin-like growth factor 1 (IGF-1) acts as a ligand for IGF1R; its binding to the alpha subunit of IGF1R, leads to activation of the intrinsic tyrosine kinase activity which autophosphorylates tyrosine residues in the beta subunit; thus, initiating a cascade of down-stream signaling events leading to activation of the PI3K-AKT/PKB and the Ras-MAPK pathways. IGF-1 also binds to integrins ITGAV:ITGB3 and ITGA6:ITGB4; its binding to integrins and subsequent ternary complex formation with integrins and IGFR1 contribute to IGF-1 signaling.

The IGF-binding proteins bind and sequester free IGF. Thus, when IGF-1 is bound to IGFBP-4 or IGFBP-5, IGF-1 is prevented from interacting with its receptor IGF1R.

Pregnancy-associated plasma protein-A (PAPP-A) is a growth-promoting metalloproteinase that cleaves insulin-like growth factor (IGF)-dependent IGF-binding protein-4 (IGFBP-4) or IGFBP-5. This releases IGF-1 and allows it to interact with its receptor, IGF1R. PAPP-A2 is also a growth-promoting metalloproteinase that cleaves IGFBP-5, which releases IGF-1 and allows it to interact with IGF1R.

Mammalian stanniocalcin-2 (STC-2) is a secreted homodimeric glycoprotein that is expressed in a wide variety of developing and adult tissues and may have autocrine or paracrine functions. STC-2 binds PAPP-A and/or PAPP-A2, which inhibits their proteolytic activity in cleaving insulin-like growth factor-binding protein (IGFBP)-4 and/or IGFBP-5. Accordingly, in the presence of STC-2, PAPP-A is unable to cleave IGFBP-4 and PAPP-A2 is unable to cleave IGFBP-5; thus, IGF-1 remains bound to its binding protein such that it is unable to interact with its receptor, IGF1R. Accordingly, overexpressing STC-2 will reduce the amount of free IGF-1 and, thus, the amount of IGF-1 signaling.

The human stc-2 gene's sequence is found at cytogenic location: 5q35.2. An STC-2 gene may comprise the following nucleotide sequence (or a variant thereof):

(SEQ ID NO: 1)
TTTCTCCTTCCCTCCACGGGCCGGGTGAGAAAGTAGCCGGGGGCTATCC
CGACCCGGCGGTTCTTGGGGAGGGGGCCGAACAAGAAAAGGGAGGAG
ATGGAGATAACTTCCCCGGATTTAGCTTTTTTGTCTTTGTTTTTGTTCTC
ACCACTTCCATCGGATGACTGGAGAGTAAAAGGGAACCCGGAGCGGGGT
GGCGAGCAGCGCTTTGAGAAAATGCAGGAGTGTGTTTGGAGACGCGTA
AAGTTGCCTTTCAAGCTCTGGCCTCCGGGCACGCGATGCTCCGCGGCGG
GCTGACTCAGGGCTGCCTTGGGCCTCCCTGCCACCCTCCTGGAAATGAT
GCAAGTCCTGACTGTCACCTGGATCCCTGCAGCCCAGCCTGGAATGCGT
CTGGATTAGGGGAAAGACGAGAAACGACACTCCAGGTGTTGCACGGCC
CACCAAAGCGGGAAGATAGGGCAGTTGCTCAGACCAAATACTGTATCT
AGTGCTTCTGCTCCTATCTTCAATCGTGGGGTTCTTTTTAATGCAAAGTG
TCACAAGGCCAGGAATTCCCATGTGTGCTCAGTTGGCCCACAGCATCAT
TGTGCCTAGGAAACTGCTTCAATTTATCAAGTCCTCTGGGCTGGGAATC
TCACTGAATTCCAAACGGCGGAAAGAGGAAACTTTCCCAACCCGATGT
GGGTGTGACGCGAGCCAGGGGCCCCAGGGACACTGTCCCAGAGCACAC
CGTCCCCCTTTAACAGCAACTGGAGCTTGGATTCGCTCTTATATTGTACA
GTCCTTTCGACCATTGCCCTGGAGCACCCGCACACGCGCACGCATCTCC
GGCCGCGCTCACACACACTCATACACACGCACGCAAACGCGTGGCCGC
CGCCAGGTCGGCAACTTTGTCCGGCGCTCCCAGCGGCGCTCGGCTTCCT
CCTGTAGTAGTTGAGCGCAGGCCCCGCCTCCCGGCCGTGTTGTCAAAAG
GGCCGGGGTCTCGGATTGGTCCAGCCGCCGGGACAACACCTGCTCGACT
CCTTCATTCAAGTGACACCAGAGCTTCCAGGGATATTTGAGGCACCATC
CCTGCCATTGCCGGGCACTCGCGGCGCTGCTAACGGCCTGGTCACATGC
TCTCCGGAGAGCTACGGGAGGGCGCTGGGTAACCTCTATCCGAGCCGC
GGCCGCGAGGAGGAGGGAAAAGGCGAGCAAAAAGGAAGAGTGGGAGG
AGGAGGGGAAGCGGCGAAGGAGGAAGAGGAGGAGGAGGAAGAGGGG
AGCACAAAGGATCCAGGTCTCCCGACGGGAGGTTAATACCAAGAACCA
TGTGTGCCGAGCGGCTGGGCCAGTTCATGACCCTGGCTTTGGTGTTGGC
CACCTTTGACCCGGCGCGGGGGACCGACGCCACCAACCCACCCGAGGG
TCCCCAAGACAGGAGCTCCCAGCAGAAAGGCCGCCTGTCCCTGCAGAA
TACAGCGGAGATCCAGCACTGTTTGGTCAACGCTGGCGATGTGGGGTGT
GGCGTGTTTGAATGTTTCGAGAACAACTCTTGTGAGATTCGGGGCTTAC
ATGGGATTTGCATGACTTTTCTGCACAACGCTGGAAAATTTGATGCCCA
GGGCAAGTCATTCATCAAAGACGCCTTGAAATGTAAGGCCCACGCTCTG
CGGCACAGGTTCGGCTGCATAAGCCGGAAGTGCCCGGCCATCAGGGAA
ATGGTGTCCCAGTTGCAGCGGGAATGCTACCTCAAGCACGACCTGTGCG
CGGCTGCCCAGGAGAACACCCGGGTGATAGTGGAGATGATCCATTTCA
AGGACTTGCTGCTGCACGAACCCTACGTGGACCTCGTGAACTTGCTGCT
GACCTGTGGGGAGGAGGTGAAGGAGGCCATCACCCACAGCGTGCAGGT
TCAGTGTGAGCAGAACTGGGGAAGCCTGTGCTCCATCTTGAGCTTCTGC
ACCTCGGCCATCCAGAAGCCTCCCACGGCGCCCCCCGAGCGCCAGCCCC
AGGTGGACAGAACCAAGCTCTCCAGGGCCCACCACGGGGAAGCAGGAC
ATCACCTCCCAGAGCCCAGCAGTAGGGAGACTGGCCGAGGTGCCAAGG
GTGAGCGAGGTAGCAAGAGCCACCCAAACGCCCATGCCCGAGGCAGAG
TCGGGGGCCTTGGGGCTCAGGGACCTTCCGGAAGCAGCGAGTGGGAAG
ACGAACAGTCTGAGTATTCTGATATCCGGAGGTGAAATGAAAGGCCTG
GCCACGAAATCTTTCCTCCACGCCGTCCATTTTCTTATCTATGGACATTC
CAAAACATTTACCATTAGAGAGGGGGGATGTCACACGCAGGATTCTGT
GGGGACTGTGGACTTCATCGAGGTGTGTGTTCGCGGAACGGACAGGTG
AGATGGAGACCCCTGGGGCCGTGGGGTCTCAGGGGTGCCTGGTGAATT
CTGCACTTACACGTACTCAAGGGAGCGCGCCCGCGTTATCCTCGTACCT
TTGTCTTCTTTCCATCTGTGGAGTCAGTGGGTGTCGGCCGCTCTGTTGTG
GGGGAGGTGAACCAGGGAGGGGCAGGGCAAGGCAGGGCCCCCAGAGC
TGGGCCACACAGTGGGTGCTGGGCCTCGCCCCGAAGCTTCTGGTGCAGC
AGCCTCTGGTGCTGTCTCCGCGGAAGTCAGGGCGGCTGGATTCCAGGAC
AGGAGTGAATGTAAAAATAAATATCGCTTAGAATGCAGGAGAAGGGTG
GAGAGGAGGCAGGGGCCGAGGGGGTGCTTGGTGCCAAACTGAAATTCA
GTTTCTTGTGTGGGGCCTTGCGGTTCAGAGCTCTTGGCGAGGGTGGAGG
GAGGAGTGTCATTTCTATGTGTAATTTCTGAGCCATTGTACTGTCTGGGC
TGGGGGGGACACTGTCCAAGGGAGTGGCCCCTATGAGTTTATATTTTAA
CCACTGCTTCAAATCTCGATTTCACTTTTTTTATTTATCCAGTTATATCT
ACATATCTGTCATCTAAATAAATGGCTTTCAAACAAAGCAACTGGGTCAT
TAAAACCAGCTCAAAGGGGGTTTAAAAAAAAAAAACCAGCCCATCCTT
TGAGGCTGATTTTTCTTTTTTTTAAGTTCTATTTTAAAAGCTATCAAACA
GCGACATAGCCATACATCTGACTGCCTGACATGGACTCCTGCCCACTTG
GGGGAAACCTTATACCCAGAGGAAAATACACACCTGGGGAGTACATTT
GACAAATTTCCCTTAGGATTTCGTTATCTCACCTTGACCCTCAGCCAAGA
TTGGTAAAGCTGCGTCCTGGCGATTCCAGGAGACCCAGCTGGAAACCTG
GCTTCTCCATGTGAGGGGATGGGAAAGGAAAGAAGAGAATGAAGACTA
CTTAGTAATTCCCATCAGGAAATGCTGACCTTTTACATAAAATCAAGGA
GACTGCTGAAAATCTCTAAGGGACAGGATTTTCCAGATCCTAATTGGAA
ATTTAGCAATAAGGAGAGGAGTCCAAGGGGACAAATAAAGGCAGAGA
GAAGAGACAGAACTAAAAATACGAGGAAAGGAGAGTGAGGATTTTCAT
TAAAAGTCTCAGCAGTGGGTTTCTTGGGTTATTTAAAACATCACCTAAA
TAGGCCTTTTCTTCCTAATTGGCCATCAAATTAAAGCCTATCCTTTCTCA
AGCAGGAGCTGGTATTGTAGGGAGTGGCCGGGTATTCTGGGCTGGGCTC
TTCTGGAGTAGGGGGTCAGCAAACATTGTCTGCAAAGGGCCAGATACT
GAATCCAGTACTTTCAGTTTGGCGAGCCGTGAGGTCTCTGTCGAAACTA
CTCAACTCTGCCGTCCTAGCACAAAAGCAGCCATAGACAACACACAAA
CGAGAGGGCTTGGCTCCCTTCCAGGAAGATTTATTTAACAGGCTCCCAG
CTGAATTTCACTCACAGGACACAGTTTACTGATCTCTGTTCTAGTGAGTG
GGTCAAAAAGCATATGCATCCTTATCCGTCAACTCATCAGCTCTTCCTC
AAGGCAACCTGAGGCCAGACACCAAGAAACCAAGCGTATCTGCTCTAA
AATGACTTGTTCCTGGGGAATGCCTTCAACCAAAACACAGCTAGTATTT
CTATGCCCCAAATCCAATCCCAGTCTTTCATGATCCATGCCGGCGGTTG
GGTGGGGAGGGGAATCATTGGTTGGGGGAAGGGAGGAAACCCCACCTC
CAGCCCCCGCCACCGGGCTCCCTGGGCACCCAGCAAGATCTGGGGCTGC
AGAGAACAGAAGAGCTGGTGCACTTAATCCAGCTCTGCCCTTGGGGGG
AGGAGGACCTGTGTGTCAGGCTCTGCCATGGGAACGAGTGTAAACCGT
GGCTGTCTCCTGCAGTGAGCCACCGCGGCAGGCACGTTGACTGTTTTAC
TGACATCACTCAAAAGCTAAAGCAATAACATTCTCCTGCGTTGCTGAGT
CAGCTGTTCATTTGTCCGCCAGCTCCTGGACTGGATGTGTGAAAGGCAT
CACATTTCCATTTTCCTCCGTGTAAATGTTTTATGTGTTCGCCTACTGAT
CCCATTCGTTGCTTCTATTGTAAATATTTGTCATTTGTATTTATTATCTC
TGTGTTTTCCCCCTAAGGCATAAAATGGTTTACTGTGTTCATTTGAACCC
ATTTACTGATCTCTGTTGTATATTTTTCATGCCACTGCTTTGTTTTCTCC
TCAGAAGTCGGGTAGATAGCATTTCTATCCCATCCCTCACGTTATTGGAA
GCATGCAACAGTATTTATTGCTCAGGGTCTTCTGCTTAAAACTGAGGAAG
GTCCACATTCCTGCAAGCATTGATTGAGACATTTGCACAATCTAAAATG
TAAGCAAAGTAGTCATTAAAAATACACCCTCTACTTGGGCTTTATACTG
CATACAAATTTACTCATGAGCCTTCCTTTGAGGAAGGATGTGGATCTCC
AAATAAAGATTTAGTGTTTATTTTGAGCTCTGCATCTTAACAAGATGATC
TGAACACCTCTCCTTTGTATCAATAAATAGCCCTGTTATTCTGAAGTGAG
AGGACCAAGTATAGTAAAATGCTGACATCTAAAACTAAATAAATAGAA
AACACCAGGCCAGAACTATAGTCATACTCACACAAAGGGAGAAATTTA
AACTCGAACCAAGCAAAAGGCTTCACGGAAATAGCATGGAAAAACAAT
GCTTCCAGTGGCCACTTCCTAAGGAGGAACAACCCCGTCTGATCTCAGA
ATTGGCACCACGTGAGCTTGCTAAGTGATAATATCTGTTTCTACTACGG
ATTTAGGCAACAGGACCTGTACATTGTCACATTGCATTATTTTTCTTCAA
GCGTTAATAAAAGTTTTAAATAAATGGCAAAAAAAAAAAAAAAAAA

A variant of the stc-2 gene may comprise a nucleotide sequence that is at least 95% identical to SEQ ID NO: 1 or to a complement thereof. As examples, at least 96%, 97%, 98%, 99%, 99.1%, 99.2%, 99.3%, 99.4%, 99.5%, 99.6%, 99.7%, 99.8%, or 99.9% identical to SEQ ID NO: 1 or to the complement thereof.

A coding sequence (CDS) for stc-2 may comprise the following nucleotide sequence (or a variant thereof):

(SEQ ID NO: 2)
ATGTGTGCCGAGCGGCTGGGCCAGTTCATGACCCTGGCTTTGGTGTTGG
CCACCTTTGACCCGGCGCGGGGGACCGACGCCACCAACCCACCCGAGG
GTCCCCAAGACAGGAGCTCCCAGCAGAAAGGCCGCCTGTCCCTGCAGA
ATACAGCGGAGATCCAGCACTGTTTGGTCAACGCTGGCGATGTGGGGTG
TGGCGTGTTTGAATGTTTCGAGAACAACTCTTGTGAGATTCGGGGCTTA
CATGGGATTTGCATGACTTTTCTGCACAACGCTGGAAAATTTGATGCCC
AGGGCAAGTCATTCATCAAAGACGCCTTGAAATGTAAGGCCCACGCTCT
GCGGCACAGGTTCGGCTGCATAAGCCGGAAGTGCCCGGCCATCAGGGA
AATGGTGTCCCAGTTGCAGCGGGAATGCTACCTCAAGCACGACCTGTGC
GCGGCTGCCCAGGAGAACACCCGGGTGATAGTGGAGATGATCCATTTC
AAGGACTTGCTGCTGCACGAACCCTACGTGGACCTCGTGAACTTGCTGC
TGACCTGTGGGGAGGAGGTGAAGGAGGCCATCACCCACAGCGTGCAGG
TTCAGTGTGAGCAGAACTGGGGAAGCCTGTGCTCCATCTTGAGCTTCTG
CACCTCGGCCATCCAGAAGCCTCCCACGGCGCCCCCCGAGCGCCAGCCC
CAGGTGGACAGAACCAAGCTCTCCAGGGCCCACCACGGGGAAGCAGGA
CATCACCTCCCAGAGCCCAGCAGTAGGGAGACTGGCCGAGGTGCCAAG
GGTGAGCGAGGTAGCAAGAGCCACCCAAACGCCCATGCCCGAGGCAGA
GTCGGGGGCCTTGGGGCTCAGGGACCTTCCGGAAGCAGCGAGTGGGAA
GACGAACAGTCTGAGTATTCTGATATCCGGAGGTGA

A variant of the stc-2 CDS may comprises a nucleotide sequence that is at least 95% identical to SEQ ID NO: 2 or to a complement thereof. As examples, at least 96%, 97%, 98%, 99%, 99.1%, 99.2%, 99.3%, 99.4%, 99.5%, 99.6%, 99.7%, 99.8%, or 99.9% identical to SEQ ID NO: 2 or to the complement thereof.

STC-2 may comprise the following amino acid sequence (or a variant thereof):

(SEQ ID NO: 3)
MCAERLGQFMTLALVLATFDPARGTDATNPPEGPQDRSSQQKGRLSLQN
TAEIQHCLVNAGDVGCGVFECFENNSCEIRGLHGICMTFLHNAGKFDAQ
GKSFIKDALKCKAHALRHRFGCISRKCPAIREMVSQLQRECYLKHDLCA
AAQENTRVIVEMIHFKDLLLHEPYVDLVNLLLTCGEEVKEAITHSVQVQ
CEQNWGSLCSILSFCTSAIQKPPTAPPERQPQVDRTKLSRAHHGEAGHH
LPEPSSRETGRGAKGERGSKSHPNAHARGRVGGLGAQGPSGSSEWEDEQ
SEYSDIRR

A variant of the STC-2 may comprise an amino acid sequence that is at least 95% identical to SEQ ID NO: 3. As examples, at least 96%, 97%, 98%, 99%, 99.1%, 99.2%, 99.3%, 99.4%, 99.5%, 99.6%, 99.7%, 99.8%, or 99.9% identical to SEQ ID NO: 3.

STC-2 has been characterized in, at least, DiMattia et al., “Molecular cloning and characterization of stanniocalcin-related protein.” Mol. Cell. Endocrinol. 146 (1-2), 137-140 (1998); Ishibashi et al., “Molecular cloning of a second human stanniocalcin homologue (STC2)” Biochem. Biophys. Res. Commun. 250 (2), 252-258 (1998); and Roch and Sherwood “Stanniocalcin Has Deep Evolutionary Roots in Eukaryotes” Genome Biol. Evol. 3:284-294 (2011); the contents of each of which is incorporated by reference in its entirety. Ten of STC-2's fifteen cysteine residues are generally conserved among stanniocalcin family members. Its serine residues are often phosphorylated by casein kinase 2 and its C-terminus contains a cluster of histidine residues which may interact with metal ions. Proteolytic inhibition of PAPP-A requires covalent binding of STC-2 to PAPP-A, which is mediated by a disulfide bond that involves STC-2's Cys-120. See, Jepsen et al, “Stanniocalcin-2 Inhibits Mammalian Growth by Proteolytic Inhibition of the Insulin-like Growth Factor Axis” J Biol Chem. 2015, 290(6):3430-9. For further descriptions of human STC-2, see, the World Wide Web (www) at ncbi.nlm.nih.gov/gene/8614 and UniProtKB/Swiss-Prot: 076061. The contents of each of which is incorporated by reference in its entirety.

The present disclosure includes fragments of STC-2 (and variants thereof) and/or nucleic acids that encode a fragment of STC-2 (and variants thereof). By fragment of STC-2 is meant a fragment of a full-length STC-2 protein sequence (e.g., SEQ ID NO: 3 or a variant thereof) that is capable of binding human PAPP-A and/or PAPP-A2 and inhibiting human PAPP-A or PAPP-A2's proteinase activity. The fragment may include a full-length human STC-2 protein sequence, e.g., SEQ ID NO: 3. Alternately, the fragment may include less than the full-length STC-2 protein sequence, e.g., is one or more amino acids shorter than the length of a human STC-2 protein sequence (e.g., SEQ ID NO: 3). As used herein, a fragment may be about 1, 2, 3, 4, 5, 6, 7, 8, 9, 10, 20, 30, 40, 50, 60, 70, 80, 90, 100, 200, or 300 amino acids (and any number in between) shorter than the full length of a human STC-2 protein sequence, as long as the fragment is capable of binding human PAPP-A and/or PAPP-A2 and inhibiting their proteinase activity. As used herein, a fragment may also be a variant of a human STC-2 protein sequence, such that the fragment is shorter than the full length of a human STC-2 protein sequence and includes amino acid changes relative to the full length human STC-2 protein sequence. In some embodiments, a variant of an STC-2 or fragment thereof retains certain cysteine residues that are generally conserved among stanniocalcin family members and especially, the cysteine corresponding to Cys-120.

The present disclosure further comprises use of non-human STC-2 (and variants thereof) and nucleic acids encoding the same, e.g., homologs of human STC-2. The term homolog, as used herein, is a gene (or its translated protein) that is inherited in two species by a common ancestor. Although homologs can be similar in sequence, similar sequences are not necessarily homologous. However, the main functions of homologs are typically conserved, at least among related species. Non-limiting examples of non-human STC-2 include:

Canis familiaris (Dog) [See, UniProtKB-J9P095]
(SEQ ID NO: 4)
MCAERLGQFVTLALVLATFDLARGTDATNPPEGPQDRGSQQKGRLSLQNT
AEIQHCLVNAGDVGCGVFECFENNSCEIRGLHGICMTFLHNAGKFDAQGKS
FIKDALKCKAHALRHRFGCISRKCPAIKEMVFQLQRECYLKHDLCSAAQEN
TRVMVEMIHFKDLLLHEPYVDLVNLLLTCGEEVKEAITHSVQAQCEQNWG
SLCSILSFCTSAIQRPPTVPPERQLQVDRAKLSKTHHGEAGHHVSEPSSWET
GRGTKGEGGSKSHLNAHARGRAVGQGGQGTSGSSEWEEEPSEYSDIRR
Felis catus (Cat) [See, UniProtKB-M3WZL4]
(SEQ ID NO: 5)
MCAERLGQFVTLALVLATFDPARGTDATNPPEGPQDRGSQQKGRLSLQNT
AEIQHCLVNAGDVGCGVFECFENNSCEIRGLHGICMTFLHNAGKFDAQGKS
FIKEALKCKAHALRHRFGCISRKCPAIKEMVFQLQRECYLKHDLCSAAQEN
TRVMVEMIHFKDLLLHEPYVDLVNLLLTCGEEVKEAITHSVQAQCEQNWG
SLCSILSFCTSAIQRPPTVPPERQLQVDRAKLSRAHHGDTGHHLSEASSRETG
RGVKGERGSKSHPNAHARGRAAGHGGQGTSGSSEWEEEQSEYSDIRR
Equus caballus (Horse) [See, UniProtKB-F6YPR7]
(SEQ ID NO: 6)
MCTERLGQFMTLALVLATFDPARGTDATHPPEGPQDRGSQQKGRLSLQNT
AEIQHCLVNAGDVGCGVFECFENNSCEIRGLHGICMTFLHNAGKFDAQGKS
FIKDALKCKAHALRHRFGCISRKCPAIKEMVFQLQRECYLKHDLCSAAQEN
TRVMVEIIHFKDLLLHEPYVDLVNLLLTCGEEVKEAITHSVQAQCEQSWGS
LCSILSFCTSAIQRPPTVPPERRLQGDRARLSRAHHEEAGHHLSEPSSQETGR
GAKGERGSKSHPNTHARGRAGGHGAQGTSGSSEWEDEQSEYSDIRR
Bos taurus (Bovine) [See, UniProtKB-E1B8P3]
(SEQ ID NO: 7)
MCAERLGQFVTLALVLVTFDPARGTDATNTPEGPQDRGSQQKGRLSLQNT
AEIQHCLVNAGDVGCGVFECFENNSCEIRGLHGICMTFLHNAGKFDAQGKS
FIKDALKCKAHALRHRFSCISRKCPAIKEMVFQLQRECYLKHDLCSAAQENI
RVMVEMIHFKDLLLHEPYVDLVNLLLTCGEEVKEAITHSVQAQCEQSWGS
LCSILSFCTSAIQRPPTAPPEHQLQGDRAKLSRGHPAEMGHHLAEPSSRETG
RGAKGERGSKSHPNAHARGRAAGPGAQGTSGSSEWEDEQSEYSDIRR
Sus scrofa (Pig) [See, UniProtKB-FISJZ1]
(SEQ ID NO: 8)
MCAERLGQFVTLALLLATFDPARGTEATNPPESPQDRGSQQKGRLSLQNTA
EIQHCLVNAGDVGCGVFECFENNSCEIRGLHGICMTFLHNAGKFDAQGKSF
IKDALKCKAHALRHRFGCISRKCPAIKEMVFQLQRECYLKHDLCSAAQENT
RVMVEMIHFKDLLLHEPYVDLVNLLLTCGEEVKEAITHSVQAQCEQNWGS
LCSILSFCTSAIQRPPTAPPEHPPQGDRAKPSRAHGEAGHPLSEPSSRETGRG
AKGERASKSHPIAHARGRAAGHGAQGTSGSSEWEDEQSEYSDIRR
Oryctolagus cuniculus (Rabbit) [See, UniProtKB-G1SU78]
(SEQ ID NO: 9)
MCAERLGRFVTLTLVFATFDLARGTDATHPPEGLPDRGSQQKGRLSLQNT
AEIQHCLVNAGDVGCGVFECFENNSCEIRGLHGICMTFLHNAGKFDAQGKS
FIKDALKCKAHALRHRFGCISRKCPAIKEMVFQLQRECYLKHDLCSAAQDN
IRVIVEMIHFKDLLLHEPYVDLVNLLLTCGEEVKEAVTHSVQAQCEQNWG
GLCSILSFCTPTIQRPPTALPERQLQVDRTKLPRAHHGEAGPHHAEPSSKETS
RGAKGERGSRSHPNAHARGRAGSPGAQGPSGSSEWEDEQSEYSDIRR
Ovis aries (Sheep) [See, UniProtKB-W5P217]
(SEQ ID NO: 10)
MCAERLGQFVTLALVLVTFDPARGTDATNTPEGPQDRGSQQKGRLSLQNT
AEIQHCLVNAGDVGCGVFECFENNSCEIRGLHGICMTFLHNAGKFDAQGKS
FIKDALKCKAHALRHRFSCISRKCPAIKEMVFQLQRECYLKHDLCSAAQENI
RVMVEMIHFKDLLLHDPTVDLVNLLLTCGEEVKEAITHSVQAQCEQSWGS
LCSILSFCTSAIQRPPTAPPERQLQGDRAKLSRGHPAEMGHHLAEPSSRETG
RGAKGERGSKSHPNAHARGRTAGPGAQGTSGSSEWEDEQSEYSDIRR
Macaca nemestrina (Pig-tailed macaque) [See, UniProtKB-O97561]
(SEQ ID NO: 11)
MCAERLGHFMTLALVLATIDPARGTDATNPPEGPQDRSSQQKGRLSLQNT
AEIQHCLVNAGDVGCGVFECFENNSCEIRGLHGICMTFLHNAGKFDAQGKS
FIKDALKCKAHALRHRFGCISRKCPAIREMVFQLQRECYLKHDLCAAAQEN
TRVIVEMIHFKDLLLHEPYVDLVNLLLTCGEEVKEAITHSVQVQCEQNWGS
LCSILSFCTSAIQRPPTAPPERQPQVDRAKLSRAHHGEAGHHLPEPSSRETGR
GAKGERGSKSHPNAHARGRVGGLGAQGPSGSSEWEDEQSEYSDIRR
Pan troglodytes (Chimpanzee) [See, UniProtKB-H2QS12]
(SEQ ID NO: 12)
MCAERLGQFMTLALVLATFDPARGTDATNPPEGPQDRSSQQKGRLSLQNT
AEIQHCLVNAGDVGCGVFECFENNSCEIRGLHGICMTFLHNAGKFDAQGKS
FIKDALKCKAHALRHRFGCISRKCPAIREMVSQLQRECYLKHDLCAAAQEN
TRVIVEMIHFKDLLLHEPYVDLVNLLLTCGEEVKEAITHSVQVQCEQNWGS
LCSILSFCTSAIQRPPTAPPERQPQVDRTKLSRAHHGEAGHHLPEPSSRETGR
GAKGERGSKSHPNAHARGRVGGLGAQGPSGSSEWEDEQSEYSDIRR
Mus musculus (Mouse) [See, UniProtKB O88452]
(SEQ ID NO: 13)
MCAERLGQFVTLALVFATLDPAQGTDSTNPPEGPQDRSSQQKGRLSLQNT
AEIQHCLVNAGDVGCGVFECFENNSCEIQGLHGICMTFLHNAGKFDAQGK
SFIKDALRCKAHALRHKFGCISRKCPAIREMVFQLQRECYLKHDLCSAAQE
NVGVIVEMIHFKDLLLHEPYVDLVNLLLTCGEDVKEAVTRSVQAQCEQSW
GGLCSILSFCTSNIQRPPTAAPEHQPLADRAQLSRPHHRDTDHHLTANRGAK
GERGSKSHPNAHARGRTGGQSAQGPSGSSEWEDEQSEYSDIRR
Rattus norvegicus (Rat) [See, UniProtKB-Q9R0K8]
(SEQ ID NO: 14)
MCAERLGQFVTLALVFATLDPARGTDSTNPPEGPQDRGSQQKGRLSLQNT
AEIQHCLVNAGDVGCGVFECFENNSCEIQGLHGICMTFLHNAGKFDAQGK
SFIKDALRCKAHALRHKFGCISRKCPAIREMVYQLQRECYLKHDLCSAAQE
NVVVIVEMIHFKDLLLHEPYVDLVNLLLTCGEDVREAVTRSVQAQCEQSW
GGLCSILSFCTSNIQRPPTAAPEHQPLADRAQLSRPYHRDTDHHLTANRGAK
GERGSKSHLHAHARGGAGGQSAQGPSGSSEWEDEQSEYSDIRR
Cavia porcellus (Guinea pig) [See, UniProtKB-A0A286Y2C5]
(SEQ ID NO: 15)
MCAERLGQFVTLALVLATFDPARGTDATNPPEGPQDRGSQQKGRLSLQNT
ADIQHCLVNAGDVGCGVFECFENNSCEIRGLHGICMTFLHNAGKFDAQGK
SFIKDALKCKAHALRHRFGCISRKCPAIKEMVFQLQRECYLKHDLCAAAQE
NTRVLVEMIHFKDLLLHEPYVDLVNLLLTCGEEVKEAISHSVQAQCEQNW
GGLCSILSFCTSAMHRPPTVSPEHQLQMDRARLSRTHQGEASHYFSEPSSRE
SIRDAKGERGSKGHSNAHARGRAGGHGAQGPSGSSEWEDEQSEYSDIRR

A variant of an above-mentioned non-human STC-2 may comprise an amino acid sequence that is at least 95% identical to any one of SEQ ID NO: 4 to SEQ ID NO: 15, or to a fragment thereof. As examples, at least 96%, 97%, 98%, 99%, 99.1%, 99.2%, 99.3%, 99.4%, 99.5%, 99.6%, 99.7%, 99.8%, or 99.9% identical to any one of SEQ ID NO: 4 to SEQ ID NO: 15, or to a fragment thereof.

In some embodiments of the present disclosure, a nucleic acid encodes a non-human STC-2, a variant thereof, or fragments thereof. As examples, the nucleic acid encodes an amino acid sequence that is at least 95% identical to any one of SEQ ID NO: 4 to SEQ ID NO: 15, or to a fragment thereof.

By fragment of a non-human STC-2 is meant a fragment of a full-length STC-2 protein sequence (e.g., one of SEQ ID NO: 4 to SEQ ID NO: 15) that is capable of binding PAPP-A and/or PAPP-A2 and inhibiting their proteinase activity. The fragment may include a full-length non-human STC-2 protein sequence, e.g., of any one of SEQ ID NO: 4 to SEQ ID NO: 15. Alternately, the fragment may include less than the full-length STC-2 protein sequence, e.g., is one or more amino acids shorter than the length of an STC-2 protein sequence (e.g., of any one of SEQ ID NO: 4 to SEQ ID NO: 15). As used herein, a fragment may be 1, 2, 3, 4, 5, 6, 7, 8, 9, 10, 20, 30, 40, 50, 60, 70, 80, 90, 100, 200, or 300 (and any number in between) shorter than the full length of an STC-2 protein sequence, as long as the fragment is capable of binding PAPP-A and/or PAPP-A2 and inhibiting their proteinase activity. As used herein, a fragment may also be a variant of an STC-2 protein sequence, such that the fragment is shorter than the full length of an STC-2 protein sequence and includes amino acid changes relative to the full length STC-2 protein sequence.

The present disclosure includes fragments of a non-human STC-2 (and variants thereof) and/or nucleic acids that encode a fragment of a non-human STC-2 (and variants thereof). The term a fragment of a non-human STC-2 is meant to be a fragment of the full-length protein (e.g., of SEQ ID NO: 4 to SEQ ID NO: 15, or a variant thereof) that is capable of binding a non-human PAPP-A and/or PAPP-A2 and inhibiting the non-human PAPP-A or PAPP-A2's proteinase activity. In some embodiments, a variant of a non-human STC-2 or fragment thereof retains certain cysteine residues that are generally conserved among stanniocalcin family members and especially, the cysteine corresponding to Cys-120 of human STC-2. In some embodiments, the fragment of STC-2 (and variants thereof) is included in a modified protein.

Additional published amino acid sequences of STC-2 (human and non-human) is found at the World Wide Web (www) at uniprot.org/uniprot/?query=STC2&sort=score; the contents of which is incorporated by reference in its entirety.

Methods for Increasing Lifespan

Aspects of the present disclosure are methods for increasing lifespan in a mature mammal in need thereof and in a non-rodent mammal in need thereof. The methods can comprise administering to the mammal a therapeutically effective amount of a composition comprising a nucleic acid encoding a target protein or a functional fragment or variant thereof. In some embodiments, the nucleic acid encodes a fragment of stanniocalcin-2 (STC-2). In some embodiments, the nucleic acid increases (e.g., overexpresses) the amount of the STC-2 fragment that is expressed by the mammal. In some embodiments, the nucleic acid encodes a peptide growth hormone receptor antagonist or a fragment thereof. In some embodiments, the nucleic acid encodes a growth hormone releasing hormone antagonists or a fragment thereof. In some embodiments, the nucleic acid encodes a peptide inhibitor of PAPP-A or a fragment thereof.

The terms overexpress, overexpression, and overexpressing, and the like, describe that a gene (e.g., stc-2 or a fragment thereof) is expressed at a level higher than normal. Normal is relative to a cell and/or a mammal, e.g., of an equivalent species, strain, breed, sex, age, weight, size, health, and/or disease status, which is not administered the nucleic acid.

The term gene, as used herein, refers to a segment of a nucleic acid that encodes an individual protein or RNA (also referred to as a coding sequence or coding region), optionally together with associated regulatory region such as promoter, operator, terminator and the like, which may be located upstream or downstream of the coding sequence.

In these aspects, a mammal is administered a composition comprising a nucleic acid that is capable of being expressed by a cell in a mammal Thus, the nucleic acid comprises domains useful for expression/overexpression of the STC-2 fragment or modified protein comprising the STC-2 fragment in the cell.

As an example, when the nucleic acid is a synthetic RNA, e.g., an mRNA, such domains include, the nucleic acid sequence encoding the target protein or a functional fragment or variant thereof (e.g., the fragment of STC-2), one or more of 5′ cap structure, a Kozak consensus sequence, a 5′-UTR, a 3′-UTR, and a 3′ poly(A) tail. Such mRNA may be administered to a mammal in a liposome or a lipid nanoparticle or in a composition lacking a liposome or a lipid nanoparticle, e.g., as naked mRNA.

In another example, the nucleic acid that provides an overexpression of a target protein or a functional fragment or variant thereof (e.g., STC-2 overexpression) is included in an expression vector. The viral expression vector may be a lentivirus or an adeno-associated virus (AAV). In some embodiments, the viral vector is an AAV selected from AAV1, AAV2, AAV3, AAV4, AAV5, AAV6, AAV7, AAV8, AAV9, AAVrh8, AAVrh8R, AAV10, AAVrh10, AAV11, and AAV12. Thus, the nucleic acid, in this embodiment, may comprise domains useful for expression/overexpression of the target protein or a functional fragment or variant thereof (e.g., STC-2 fragment) in the cell. More specifically, the nucleic acid, e.g., a DNA, comprises a promoter selected from a CMV, CAG, CBA, EF1a, RSV, MSCV, ALB, or TBG promoter sequence and a termination and/or polyadenylation sequence selected from an SV40, hGH, BGH, or rbGlob termination and/or polyadenylation sequence.

As used herein, expression at a level higher than normal comprises any increased expression. The terms increased, increase, or increasing, and the like, are used herein to generally mean an increase by a measurable amount, e.g., a statistically significant amount, over the normal level of expression. As examples, the increase may be 1% to 1000% increased expression or more over the normal level of expression. In some embodiments, the increase is of at least about 10%, at least about 20%, or at least about 30%, or at least about 40%, or at least about 50%, or at least about 60%, or at least about 70%, or at least about 80%, or at least about 90% or up to and including a 100% increase, or a greater increase over the normal level of expression. Other examples of increase include an increase of at least 2-fold, at least 5-fold, at least 10-fold, at least 20-fold, at least 50-fold, at least 100-fold, at least 1000-fold or more over the normal level of expression. When the cell does not normally express the STC-2 fragment (or modified protein), the increased expression is incalculable and any expression of the STC-2 fragment (or modified protein) comprises an increase. Methods for determining and/or quantifying overexpression are well-known in the art.

In some embodiments, administering the composition comprises intravenous injection or infusion, intraperitoneal injection, intramuscular injection, or subcutaneous injection. In some embodiments comprising an expression vector, the administering is not repeated or is not repeated for at least five years after initial administering. In some embodiments not comprising an expression vector, the administering is repeated at least one additional time, e.g., two, three, four, five, ten, or more times.

In some embodiments, the overexpressed STC-2 fragment (either alone or in a modified protein) is capable of binding pregnancy-associated plasma protein-A (PAPP-A) and/or PAPP-A2 and inhibiting their enzymatic activity.

Nucleic Acid—Synthetic RNA

In compositions and methods of the present disclosure, a nucleic acid encoding a target protein or a functional fragment or variant thereof (e.g., an STC-2 fragment or variant thereof) or encoding a modified protein comprising a target protein or a functional fragment or variant thereof (e.g., an STC-2 fragment or variant thereof) is a synthetic RNA. Any non-natural RNA of the present disclosure may be understood to be a synthetic RNA. In some embodiments, the synthetic RNA is an mRNA. In some embodiments, the nucleic acid encodes a peptide growth hormone receptor antagonist or a fragment thereof. In some embodiments, the nucleic acid encodes a growth hormone releasing hormone antagonists or a fragment thereof. In some embodiments, the nucleic acid encodes a peptide inhibitor of PAPP-A or a fragment thereof.

Nucleic acids described herein may be of various lengths, generally dependent upon the particular form of nucleic acid. For example, in some embodiments, plasmids or genes may be from about 1,000 to about 100,000 nucleotide residues in length. In some embodiments, oligonucleotides may range from about 10 to about 100 nucleotides in length. In some embodiments, oligonucleotides, both single-stranded, double-stranded, and triple-stranded, may range in length from about 100 to about 500 nucleotides, from about 50 to about 200 nucleotides, from about 10 to about 60 nucleotides, from about 15 to about 60 nucleotides, from about 20 to about 50 nucleotides, from about 15 to about 30 nucleotides, or from about 20 to about 30 nucleotides in length.

A synthetic RNA may be transcribed using any method (or kit) known in the art. For example, a commercially-available kit or components thereof may be used to synthesize RNA. In one example, a DNA template may be transcribed using the T7 High Yield RNA Synthesis Kit (New England Biolabs, Inc.), according to the manufacturer's instructions. Synthetic RNA can be diluted with nuclease-free water and an RNase inhibitor (e.g., SuperaseIn, Life Technologies Corporation) may be added.

In some embodiments, the synthetic RNA may comprise one or more non-canonical nucleotides. In some embodiments, the one or more non-canonical nucleotides avoids substantial cellular toxicity. Examples of non-canonical nucleotides include one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides. A synthetic RNA may include one type of non-canonical nucleotide to replace a specific nucleotide, e.g., all cytidines may be replaced with 5-methylcytidine. Alternately, a synthetic RNA may include a mix of natural nucleotides and non-canonical nucleotides; the non-canonical nucleotides may be of one type or more than one type, e.g., only 5-methylcytidine and a mixture of 5-methylcytidine and 5-hydroxymethylcytidine. A synthetic RNA may have non-canonical nucleotides replacing other natural nucleotides. For example, a synthetic RNA may have all or some of its uridines replaced with non-canonical uridine residues and all or some of its cytidines replaced with non-canonical cytidines residues.

In some embodiments, the synthetic RNA may comprise a 5′ cap structure. In some embodiments, the synthetic RNA may comprise a Kozak consensus sequence. The synthetic RNA may comprise or further comprise a 5′-UTR which comprises a sequence that increases RNA stability in vivo, and the 5′-UTR optionally comprises an alpha-globin or beta-globin 5′-UTR. The synthetic RNA may comprise or further comprise a 3′-UTR which comprises a sequence that increases RNA stability in vivo, and the 3′-UTR optionally comprises an alpha-globin or beta-globin 3′-UTR. The synthetic RNA may comprise or further comprise a 3′ poly(A) tail. These additions to a synthetic RNA may be included in the DNA sequence encoding the RNA. Where appropriate, these additions may be added using any method (or kit) known in the art. For example, a commercially-available kit or components thereof may be used.

Synthetic RNA encoding a target protein or a functional fragment or variant thereof (e.g., an STC-2 fragment or variant thereof) or encoding a modified protein comprising a target protein or a functional fragment or variant thereof (e.g., an STC-2 fragment or variant thereof) is delivered in composition comprising a lipid nanoparticle or liposome or in a composition lacking a liposome or a lipid nanoparticle, e.g., as naked mRNA.

Liposomes and Lipid Nanoparticles

In some embodiments, a nucleic acid (e.g., a synthetic RNA encoding a target protein or a functional fragment or variant thereof or encoding a modified protein comprising a target protein or a functional fragment or variant thereof) or a modified protein comprising an a target protein or a functional fragment or variant thereof is delivered in a lipid nanoparticle or liposome.

A lipid nanoparticle or liposome can comprise an active agent or therapeutic agent (e.g., nucleic acid), a cationic lipid, a non-cationic lipid, and a conjugated lipid that inhibits aggregation of particles. In some embodiments, the active agent or therapeutic agent is fully encapsulated within the lipid portion of the lipid particle such that the active agent or therapeutic agent in the lipid particle is resistant in aqueous solution to enzymatic degradation, e.g., by a nuclease or protease. In some embodiments, the lipid particles described herein are substantially non-toxic to mammals such as humans. The lipid particles typically have a mean diameter of from about 40 nm to about 150 nm, from about 50 nm to about 150 nm, from about 60 nm to about 130 nm, from about 70 nm to about 110 nm, or from about to about 90 nm.

In some embodiments, the lipid particles or liposomes are serum-stable nucleic acid-lipid particles which comprise an mRNA, a cationic lipid, a non-cationic lipid (e.g., cholesterol alone or mixtures of one or more phospholipids and cholesterol), and a conjugated lipid that inhibits aggregation of the particles (e.g., one or more PEG-lipid conjugates). The lipid particle or liposome may comprise at least 1, 2, 3, 4, 5, 6, 7, 8, 9, 10, or more unmodified and/or modified RNA molecules. Lipid particles or liposomes and their method of preparation are described in, e.g., U.S. Pat. Nos. 5,753,613; 5,785,992; 5,976,567; 5,981,501; 6,110,745; and 6,320,017; and PCT Publication No. WO 96/40964, the disclosures of which are each herein incorporated by reference in their entirety for all purposes.

A lipid nanoparticle or liposome can refer to an entity containing amphiphilic molecules, hydrophobic molecules, or a mixture thereof, that is at least transiently stable in an aqueous environment, by way of non-limiting example, a micelle, a unilamellar bilayer with aqueous interior, a multilamellar bilayer, a lipid nanoparticle, any of the foregoing complexed with one or more nucleic acids, or a stable nucleic acid lipid particle. In some embodiments, a lipid nanoparticle or liposome encapsulates a nucleic acid or modified protein comprising a target protein or a functional fragment or variant thereof. In some embodiments, a lipid nanoparticle or liposome encapsulates an mRNA.

Lipid nanoparticles and liposomes can comprise one or more lipids and/or polymers that enhance uptake (e.g., encapsulation) of their cargo (protein or nucleic acid) by cells. See, e.g., Prui et al., Crit Rev Ther Drug Carrier Syst., 2009; 26(6): 523-580; Wakasar, J Drug Target, 2018, 26(4):311-318, Langer, 1990, Science 249:1527-1533; Treat et al., in “Liposomes in the Therapy of Infectious Disease and Cancer”, Lopez-Berestein and Fidler (eds.), Liss, New York, pp. 353-365 (1989); the contents of each of which is incorporated herein by reference in its entirety.

In some embodiments, lipid nanoparticles and liposomes include lipids selected from one or more of the following categories: cationic lipids; anionic lipids; neutral lipids; multi-valent charged lipids; and zwitterionic lipids. In some cases, a cationic lipid may be used to facilitate a charge-charge interaction with nucleic acids.

In some embodiments, a composition including a nucleic acid comprises a cationic liposome and/or cationic polymer formulation.

In some embodiments, a composition including a modified protein comprises a cationic liposome and/or cationic polymer formulation.

In some embodiments, the liposome further comprises a PEGylated lipid.

In some embodiments, lipid nanoparticles and liposomes include lipids selected from one or more of the following categories: cationic lipids; anionic lipids; neutral lipids; multi-valent charged lipids; and zwitterionic lipids. In some embodiments, a neutral lipid comprises a phospholipid and/or cholesterol or derivative thereof. In some cases, a cationic lipid can be used to facilitate a charge-charge interaction with nucleic acids.

In some embodiments, a lipid nanoparticle or liposome comprises the nucleic acid and at least one of the following: a cationic lipid, a phospholipid, a cholesterol or a derivative thereof, and/or a conjugated lipid that inhibit aggregation of particles. In some embodiments, a lipid nanoparticle or liposome comprises the nucleic acid and more than one of the following: a cationic lipid, a phospholipid, a cholesterol or a derivative thereof, and/or a conjugated lipid that inhibit aggregation of particles. In some embodiments, a lipid nanoparticle or liposome comprises the nucleic acid and at least two of the following: a cationic lipid, a phospholipid, a cholesterol or a derivative thereof, and/or a conjugated lipid that inhibit aggregation of particles. In some embodiments, a lipid nanoparticle or liposome comprises the nucleic acid and at least three of the following: a cationic lipid, a phospholipid, a cholesterol or a derivative thereof, and/or a conjugated lipid that inhibit aggregation of particles. In some embodiments, a lipid nanoparticle or liposome comprises the nucleic acid and a cationic lipid, a phospholipid, a cholesterol or a derivative thereof, and a conjugated lipid that inhibit aggregation of particles.

In some embodiments, a lipid nanoparticle or liposome comprises the nucleic acid and a cationic liposome, and/or cationic polymer formulation, and/or cationic lipid. In some embodiments, a lipid nanoparticle or liposome comprises the nucleic acid and a cationic lipid.

In some embodiments, the cationic lipid comprises from about 0 to 100 mol % of total lipid present in the lipid nanoparticle or liposome. In some embodiments, the cationic lipid comprises from about 0 to 90 mol %, 0 to 80 mol %, 0 to 70 mol %, 0 to 60 mol %, 0 to 50 mol %, 0 to 40 mol %, 0 to mol %, 0 to 20 mol %, 0 to 10 mol %, 10 to 90 mol %, 20 to 90 mol %, 30 to 90 mol %, 40 to 90 mol %, 50 to 90 mol %, 60 to 90 mol %, 70 to 90 mol %, 80 to 90 mol %, 20 to 80 mol %, 30 to 70 mol %, 40 to 60 mol %, or 50 to 60 mol % of total lipid present in the lipid nanoparticle or liposome. In some embodiments, the cationic lipid comprises 30 to 90 mol % of total lipid present in the lipid nanoparticle or liposome. In some embodiments, the cationic lipid comprises 40 to 70 mol % of total lipid present in the lipid nanoparticle or liposome. In some embodiments, the cationic lipid comprises to 60 mol % of total lipid present in the lipid nanoparticle or liposome. In some embodiments, the cationic lipid comprises 50 to 60 mol % of total lipid present in the lipid nanoparticle or liposome. In some embodiments, the cationic lipid comprises 45 to 55 mol % of total lipid present in the lipid nanoparticle or liposome.

In some embodiments, the cationic lipid may comprise, e.g., one or more of the following: 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethy laminopropane chloride salt (DLin-TMA. CD, 1,2-dilinoleoyl-3-trimethy laminopropane chloride salt (DLin-TAP.C1), 1,2-dilinoley loxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido) ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis, cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof.

In some embodiments, a lipid nanoparticle or liposome comprises a cationic lipid selected from N-[1-(2,3-dioleoyloxy)propyl]-N,N,N-trimethyl ammonium salts, e.g. the methylsulfate salt. In some embodiments, a lipid nanoparticle or liposome comprises a cationic lipid selected from the family of -TAP (trimethylammonium methylsulfate), e.g., DOTAP (dioleoyl-), DOTAP (dimyristoyl-), DPTAP (dipalmitoyl-), or DSTAP (distearoyl-). In some embodiments, a lipid nanoparticle or liposome comprises a cationic lipid selected from DDAB, dimethyldioctadecyl ammonium bromide; 1,2-diacyloxy-3-trimethylammonium propanes, (including but not limited to: dioleoyl, dimyristoyl, dilauroyl, dipalmitoyl and distearoyl; also two different acyl chains can be linked to the glycerol backbone); N-[1-(2,3-dioloyloxy)propyl]-N,N-dimethyl amine (DODAP); 1,2-diacyloxy-3-dimethylammonium propanes, (including but not limited to: dioleoyl, dimyristoyl, dilauroyl, dipalmitoyl and distearoyl; also two different acyl chain can be linked to the glycerol backbone); N-[1-(2,3-dioleyloxy)propyl]-N,N,N-trimethylammonium chloride (DOTMA); 1,2-dialkyloxy-3-dimethylammonium propanes, (including but not limited to: dioleyl, dimyristyl, dilauryl, dipalmityl and distearyl; also two different alkyl chain can be linked to the glycerol backbone); dioctadecylamidoglycylspermine (DOGS); 3β-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA); β-alanyl cholesterol; cetyl trimethyl ammonium bromide (CTAB); diC14-amidine; N-tert-butyl-N′-tetradecyl-3-tetradecylamino-propionamidine; 14Dea2; N-(alpha-trimethylammonioacetyl)didodecyl-D-glutamate chloride (TMAG); O,O′-ditetradecanoyl-N-(trimethylammonio-acetyl)diethanolamine chloride; 1,3-dioleoyloxy-2-(6-carboxy-spermyl)-propylamide (DOSPER); N,N,N′,N′-tetramethyl-N,N′-bis (2-hydroxylethyl)-2,3-dioleoyloxy-1,4-butanediammonium iodide; 1-[2-(acyloxy)ethyl]2-alkyl(alkenyl)-3-(2-hydroxyethyl)-imidazolinium chloride derivatives such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy) ethyl]-2-pentadecyl-3-(2-hydroxyethyl) imidazolinium chloride (DPTIM), 2,3-dialkyloxypropyl quaternary ammonium compound derivatives, containing a hydroxyalkyl moiety on the quaternary amine such as: 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE); cationic esters of acyl carnitines as reported by Santaniello et al. (U.S. Pat. No. 5,498,633); cationic triesters of phosphatidylcholine, i.e. 1,2-diacyl-sn-glycerol-3-ethylphosphocholines, where the hydrocarbon chains can be saturated or unsaturated and branched or non-branched with a chain length from C12 to C24, the two acyl chains being not necessarily identical.

In some embodiments, a lipid nanoparticle or liposome comprises the nucleic acid and a cholesterol or derivative thereof. Non-limiting examples of cholesterol derivatives include, but are not limited to, cholestanol, cholestanone, cholestenone, coprostanol, cholesteryl-2′-hydroxyethyl ether, cholesteryl-4′-hydroxybutyl ether, or mixtures thereof.

In some embodiments, the cholesterol or derivative thereof comprises from about 0 to 100 mol % of total lipid present in the lipid nanoparticle or liposome. In some embodiments, the cholesterol or derivative thereof may comprise from about 0 to 90 mol %, 0 to 80 mol %, 0 to 70 mol %, 0 to 60 mol %, 0 to 50 mol %, 0 to 40 mol %, 0 to 30 mol %, 0 to 20 mol %, 0 to 10 mol %, 10 to 90 mol %, 20 to 90 mol %, 30 to 90 mol %, 40 to 90 mol %, 50 to 90 mol %, 60 to 90 mol %, 70 to 90 mol %, 80 to mol %, 20 to 80 mol %, 30 to 70 mol %, 40 to 60 mol %, or 50 to 60 mol % of total lipid present in the lipid nanoparticle or liposome. In some embodiments, the cholesterol or derivative thereof comprises 10 to 50 mol % of total lipid present in the lipid nanoparticle or liposome. In some embodiments, the cholesterol or derivative thereof comprises 5 to 20 mol % of total lipid present in the lipid nanoparticle or liposome. In some embodiments, the cholesterol or derivative thereof comprises to 30 mol % of total lipid present in the lipid nanoparticle or liposome. In some embodiments, the cholesterol or derivative thereof comprises 30 to 50 mol % of total lipid present in the lipid nanoparticle or liposome. In some embodiments, the cholesterol or derivative thereof comprises 35 to 45 mol % of total lipid present in the lipid nanoparticle or liposome. In some embodiments, the cholesterol or derivative thereof comprises 45 to 55 mol % of total lipid present in the lipid nanoparticle or liposome.

In some embodiments, a lipid nanoparticle or liposome comprises the nucleic acid and a phospholipid. In some embodiments, the phospholipid comprises from about 0 to 100 mol % of total lipid present in the lipid nanoparticle or liposome. In some embodiments, the phospholipid may comprise from about 0 to 90 mol %, 0 to 80 mol %, 0 to 70 mol %, 0 to 60 mol %, 0 to 50 mol %, 0 to mol %, 0 to 30 mol %, 0 to 20 mol %, 0 to 10 mol %, 10 to 90 mol %, 20 to 90 mol %, 30 to 90 mol %, 40 to 90 mol %, 50 to 90 mol %, 60 to 90 mol %, 70 to 90 mol %, 80 to 90 mol %, 20 to 80 mol %, to 70 mol %, 40 to 60 mol %, or 50 to 60 mol % of total lipid present in the lipid nanoparticle or liposome. In some embodiments, the phospholipid comprises 2 to 30 mol % of total lipid present in the lipid nanoparticle or liposome. In some embodiments, the phospholipid comprises 5 to 25 mol % of total lipid present in the lipid nanoparticle or liposome. In some embodiments, the phospholipid comprises 10 to 20 mol % of total lipid present in the lipid nanoparticle or liposome. In some embodiments, the phospholipid comprises 1 to 10 mol % of total lipid present in the lipid nanoparticle or liposome. In some embodiments, the phospholipid comprises 2 to 8 mol % of total lipid present in the lipid nanoparticle or liposome.

Non-limiting examples of phospholipid can be dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), or mixtures thereof.

In some embodiments, a lipid nanoparticle or liposome comprises the nucleic acid and a conjugated lipid that inhibit aggregation of particles. In some embodiments, the conjugated lipid that inhibit aggregation of particles may comprise from about 0 to 100 mol % of total lipid present in the lipid nanoparticle or liposome. In some embodiments, the conjugated lipid that inhibit aggregation of particles may comprise from about 0 to 90 mol %, 0 to 80 mol %, 0 to 70 mol %, 0 to 60 mol %, 0 to 50 mol %, 0 to 40 mol %, 0 to 30 mol %, 0 to 20 mol %, 0 to 10 mol %, 10 to 90 mol %, 20 to 90 mol %, to 90 mol %, 40 to 90 mol %, 50 to 90 mol %, 60 to 90 mol %, 70 to 90 mol %, 80 to 90 mol %, 20 to 80 mol %, 30 to 70 mol %, 40 to 60 mol %, or 50 to 60 mol % of total lipid present in the lipid nanoparticle or liposome. In some embodiments, the conjugated lipid comprises 5 to 50 mol % of total lipid present in the lipid nanoparticle or liposome. In some embodiments, the conjugated lipid comprises to 10 mol % of total lipid present in the lipid nanoparticle or liposome. In some embodiments, the conjugated lipid comprises 0.1 to 5 mol % of total lipid present in the lipid nanoparticle or liposome. In some embodiments, the conjugated lipid comprises 0.5 to 2.5 mol % of total lipid present in the lipid nanoparticle or liposome. In some embodiments, the conjugated lipid comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) mol % of total lipid present in the lipid nanoparticle or liposome.

In some embodiments, a lipid nanoparticle or liposome comprises either a PEG-lipid conjugate or an ATTA-lipid conjugate. Suitable conjugated lipids include, but are not limited to, PEG-lipid conjugates, ATTA-lipid conjugates, cationic-polymer-lipid conjugates (CPLs), and mixtures thereof. In some embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-dilauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the present disclosure include, but are not limited to, mPEG2000-1,2-di-O-alkyl-sn3-carbomoylglyceride (PEG-C-DOMG). The synthesis of PEG-C-DOMG is described in PCT Application No. PCT/US08/88676, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-w-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

In certain embodiments, the particles comprise either a PEG-lipid conjugate or an ATTA-lipid conjugate together with a CPL. In some embodiments, the lipid conjugate is a PEG-lipid. Examples of PEG-lipids include, but are not limited to, PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides as described in, e.g., U.S. Pat. No. 5,885,613, PEG conjugated to cholesterol or a derivative thereof, and mixtures thereof. The disclosures of these patent documents are herein incorporated by reference in their entirety for all purposes. Additional PEG-lipids include, without limitation, PEG-C-DOMG, 2 KPEG-DMG, and a mixture thereof.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene gly col-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present invention. The disclosures of these patents are herein incorporated by reference in their entirety for all purposes. In addition, monomethoxypolyethyleneglycolacetic acid (MePEG-CH2COOH) can be useful for preparing PEG-lipid conjugates including, e.g., PEG-DAA conjugates.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In some embodiments, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In some embodiments, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH2CH2C(O)—), succinamidyl (—NHC(O)CH2CH2C(O)NH—), ether, disulphide, as well as combinations thereof (such as a linker containing both a carbamate linker moiety and an amido linker moiety). In some embodiments, a carbamate linker is used to couple the PEG to the lipid.

In some embodiments, the conjugated lipid that inhibits aggregation of particles is a CPL that has the formula: A-W—Y, wherein A is a lipid moiety, W is a hydrophilic polymer, and Y is a polycationic moiety. W may be a polymer selected from the group consisting of polyethyleneglycol (PEG), polyamide, polylactic acid, polyglycolic acid, polylactic acid/polyglycolic acid copolymers, or combinations thereof, the polymer having a molecular weight of from about 250 to about 7000 daltons. In some embodiments, Y has at least 4 positive charges at a selected pH. In some embodiments, Y may be lysine, arginine, asparagine, glutamine, derivatives thereof, or combinations thereof.

In some embodiments, a lipid nanoparticle or liposome comprises a non-cationic lipid. The non-cationic lipids can be any of a variety of neutral uncharged, zwitterionic, or anionic lipids capable of producing a stable complex. Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids can be acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof.

In some embodiments, the non-cationic lipid present in the lipid particles comprises or consists of cholesterol or a derivative thereof, e.g., a phospholipid-free lipid particle formulation. In some embodiments, the non-cationic lipid present in the lipid particles comprises or consists of one or more phospholipids, e.g., a cholesterol-free lipid particle formulation. In some embodiments, the non-cationic lipid present in the lipid particles comprises or consists of a mixture of one or more phospholipids and cholesterol or a derivative thereof.

Other examples of non-cationic lipids suitable for use in the present disclosure include nonphosphorous containing lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid comprises from about 13 mol % to about 49.5 mol %, from about 20 mol % to about 45 mol %, from about 25 mol % to about 45 mol %, from about 30 mol % to about 45 mol %, from about 35 mol % to about 45 mol %, from about 20 mol % to about 40 mol %, from about 25 mol % to about 40 mol %, or from about 30 mol % to about 40 mol % of the total lipid present in the particle.

In some embodiments, the cholesterol present in phospholipid-free lipid particles comprises from about 30 mol % to about 45 mol %, from about 30 mol % to about 40 mol %, from about 35 mol % to about 45 mol %, or from about 35 mol % to about 40 mol % of the total lipid present in the particle. As a non-limiting example, a phospholipid-free lipid particle may comprise cholesterol at about 37 mol % of the total lipid present in the particle.

some embodiments, the cholesterol present in lipid particles containing a mixture of phospholipid and cholesterol comprises from about 30 mol % to about 40 mol %, from about 30 mol % to about 35 mol %, or from about 35 mol % to about 40 mol % of the total lipid present in the particle. As a non-limiting example, a lipid particle comprising a mixture of phospholipid and cholesterol may comprise cholesterol at about 34 mol % of the total lipid present in the particle.

some embodiments, the cholesterol present in lipid particles containing a mixture of phospholipid and cholesterol comprises from about 10 mol % to about 30 mol %, from about 15 mol % to about 25 mol %, or from about 17 mol % to about 23 mol % of the total lipid present in the particle. As a non-limiting example, a lipid particle comprising a mixture of phospholipid and cholesterol may comprise cholesterol at about 20 mol % of the total lipid present in the particle.

In some embodiments, where the lipid particles contain a mixture of phospholipid and cholesterol or a cholesterol derivative, the mixture may comprise up to about 40, 45, 50, 55, or 60 mol % of the total lipid present in the particle. In some embodiments, the phospholipid component in the mixture may comprise from about 2 mol % to about 12 mol %, from about 4 mol % to about 10 mol %, from about 5 mol % to about 10 mol %, from about 5 mol % to about 9 mol %, or from about 6 mol % to about 8 mol % of the total lipid present in the particle. As a non-limiting example, a lipid particle comprising a mixture of phospholipid and cholesterol may comprise a phospholipid such as DPPC or DSPC at about 7 mol % (e.g., in a mixture with about 34 mol % cholesterol) of the total lipid present in the particle. In some embodiments, the phospholipid component in the mixture may comprise from about 10 mol % to about 30 mol %, from about 15 mol % to about 25 mol %, or from about 17 mol % to about 23 mol % of the total lipid present in the particle. As another non-limiting example, a lipid particle comprising a mixture of phospholipid and cholesterol may comprise a phospholipid such as DPPC or DSPC at about 20 mol % (e.g., in a mixture with about 20 mol % cholesterol) of the total lipid present in the particle.

A liposomes or lipid nanoparticle described herein can have different sizes, lamellarity and structure. In some embodiments, the liposomes or lipid nanoparticles have an average diameter Zaverage of about 50 nm to about 500 nm. In some embodiments, the liposomes or lipid nanoparticles have a size Zaverage of about 100 to about 200 nm. The liposomes or lipid nanoparticles may be uni-, oligo- or multilamellar liposomes. In some embodiments, the liposomes or lipid nanoparticles are unilamellar liposomes.

In some embodiments, the present disclosure provides a lipid particle composition comprising a plurality of lipid particles. In some embodiments, the active agent or therapeutic agent (e.g., nucleic acid) is fully encapsulated within the lipid portion of the lipid particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the lipid particles have the active agent or therapeutic agent (e.g., a nucleic acid) encapsulated therein.

Typically, the lipid particles of the disclosure have a lipid:active agent (e.g., lipid:nucleic acid) ratio (mass/mass ratio) of from about 1 to about 100. In some instances, the lipid:active agent (e.g., lipid:nucleic acid) ratio (mass/mass ratio) ranges from about 1 to about 50, from about 2 to about 25, from about 3 to about 20, from about 4 to about 15, or from about 5 to about 10. In some embodiments, the lipid particles have a lipid:active agent (e.g., lipid:nucleic acid) ratio (mass/mass ratio) of from about to about 15, e.g., about 5, 6, 7, 8, 9, 10, 11, 12, 13, 14, or 15 (or any fraction thereof or range therein).

In some embodiments, the nucleic acid to lipid ratios (mass/mass ratios) in a formed lipid particle ranges from about 0.01 to about 0.2, from about 0.02 to about 0.1, from about 0.03 to about 0.1, or from about 0.01 to about 0.08. The ratio of the starting materials can also fall within this range. In some embodiments, the preparation uses about 400 μg nucleic acid per 10 mg total lipid or a nucleic acid to lipid mass ratio of about 0.01 to about 0.08 and. In some embodiments, the particle has a nucleic acid:lipid mass ratio of about 0.08. In some embodiments, the lipid to nucleic acid ratios (mass/mass ratios) in a formed lipid particle ranges from about 1 (1:1) to about 100 (100:1), from about 5 (5:1) to about 100 (100:1), from about 1 (1:1) to about 50 (50:1), from about 2 (2:1) to about 50 (50:1), from about 3 (3:1) to about 50 (50:1), from about 4 (4:1) to about 50 (50:1), from about 5 (5:1) to about 50 (50:1), from about 1 (1:1) to about 25 (25:1), from about 2 (2:1) to about 25 (25:1), from about 3 (3:1) to about 25 (25:1), from about 4 (4:1) to about 25 (25:1), from about 5 (5:1) to about 25 (25:1), from about 5 (5:1) to about 20 (20:1), from about 5 (5:1) to about 15 (15:1), from about 5 (5:1) to about 10 (10:1), about 5 (5:1), 6 (6:1), 7 (7:1), 8 (8:1), 9 (9:1), (10:1), 11 (11:1), 12 (12:1), 13 (13:1), 14 (14:1), or 15 (15:1). The ratio of the starting materials can also fall within this range.

Typically, the lipid particles have a mean diameter of from about 40 nm to about 200 nm. In some embodiments, the lipid particles have a mean diameter of from about 40 nm to about 150 nm, from about 40 nm to about 120 nm, from about 40 nm to about 100 nm, from about 50 nm to about 120 nm, from about 50 nm to about 100 nm, from about 60 nm to about 120 nm, from about 60 nm to about 110 nm, from about 60 nm to about 100 nm, from about 60 nm to about 90 nm, from about 60 nm to about 80 nm, from about 70 nm to about 120 nm, from about 70 nm to about 110 nm, from about 70 nm to about 100 nm, from about 70 nm to about 90 nm, from about 70 nm to about 80 nm, or less than about 120 nm, 110 nm, 100 nm, 90 nm, or 80 nm (or any fraction thereof or range therein).

In some embodiments, the nucleic acid or modified protein included in a composition comprises a Lipofectamine reagent (Life Technologies Corporation), e.g., Lipofectamine 2000, Lipofectamine 3000, Lipofectamine Stem, Lipofectamine RNAiMAX, and Invivofectamine 3.0.

Using Lipofectamine reagents as an example, nucleic acid or modified protein and a Lipofectamine transfection reagent are diluted separately in a suitable complexation medium, mixed, and incubated together, according to the manufacturer's instructions.

Liposome preparation can be made according to standard protocols. For examples, suitable lipids are diluted from stocks in ethanol to a desired concentration. The nucleic acid or modified protein is diluted to an appropriate concentration. Both solutions are transferred to syringes and mixed, e.g., using a Nanoassemblr Benchtop (Precision Nanosystems) as directed by the manufacturer. The resulting liposomes may then be formulated for in vitro or in vivo uses.

The composition, preparation and use of lipid particles or liposomes are further described herein in U.S. Pat. Nos. 8,058,069, 9,364,435, 9,364,435, 8,822,668, 9,404,127, 9,518,272B2, U.S. Ser. No. 10/626,393B2, U.S. Pat. No. 9,394,234B2, U.S. Pat. No. 9,878,042B2, U.S. Pat. No. 9,738,593B2, U.S. Ser. No. 10/106,490B2, U.S. Ser. No. 10/221,127B2, U.S. Ser. No. 10/166,298B2, U.S. Pat. No. 9,867,888B2, U.S. Ser. No. 10/266,485, U.S. Ser. No. 10/442,756, U.S. Pat. No. 9,533,047B2, U.S. Ser. No. 10/385,106, U.S. Pat. No. 8,236,770B2, U.S. Pat. No. 7,371,404B2, U.S. Pat. No. 9,066,867B2, U.S. Pat. No. 9,415,109B2, U.S. Pat. No. 9,687,448B2, the disclosures of which are each herein incorporated by reference.

Viral Expression Vector-Based Delivery of Nucleic Acids

In some embodiments, a composition of the present disclosure (or method using same) comprises a viral expression vector.

In some embodiments, the viral expression vector comprises a nucleic acid encoding a target protein or a functional fragment or variant thereof (e.g., an STC-2 fragment or variant thereof) or encoding a modified protein comprising a target protein or a functional fragment or variant thereof (an STC-2 fragment or variant thereof). The nucleic acid may be RNA or DNA. In some embodiments, the nucleic acid encodes a peptide growth hormone receptor antagonist or a fragment thereof. In some embodiments, the nucleic acid encodes a growth hormone releasing hormone antagonists or a fragment thereof. In some embodiments, the nucleic acid encodes a peptide inhibitor of PAPP-A or a fragment thereof.

Many viral expression vectors useful for gene therapy are known (see, e.g., Lundstrom, Trends Biotechnol., 21: 117, 122, 2003. Illustrative viral expression vectors include those selected from Antiviruses (LV), retroviruses (RV), adenoviruses (AV), adeno-associated viruses (AAV), and a viruses, although other viral vectors may also be used. In some in vivo uses, viral expression vectors that do not integrate into the host genome are suitable, such as a viruses and adenoviruses. Illustrative types of a viruses include Sindbis virus, Venezuelan equine encephalitis (VEE) virus, and Semliki Forest virus (SFV). For other uses, viral vectors that integrate into the host genome are suitable, such as retroviruses, AAV, and Antiviruses.

The term “adeno-associated virus,” or “AAV” as used herein refers to the adeno-associated virus or derivatives thereof. Non-limited examples of AAV's include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. In some instances, the AAV is described as a “Primate AAV,” which refers to AAV that infect primates. Likewise, an AAV may infect bovine animals (e.g., “bovine AAV”, and the like). In some instances, the AAV is wildtype, or naturally occurring. In some instances, the AAV is recombinant. Put another way, the AAV may be a variant of a naturally-occurring AAV1, AAV2, AAV3, AAV4, AAV5, AAV6, AAV7, AAV8, AAV9, AAV10, AAV11, or AAV12. By an AAV variant, it is meant an AAV having a sequence identity of 70% or more to AAV1, AAV2, AAV3, AAV4, AAV5, AAV6, AAV7, AAV8, AAV9, AAV10, AAV11, or AAV12, for example, a sequence identity of 80%, 85%, or 90% or more; of 91%, 92%, 93%, 94%, 95% or more, in some instances of 96%, 97%, 98%, or 99% to AAV1, AAV2, AAV3, AAV4, AAV5, AAV6, AAV7, AAV8, AAV9, AAV10, AAV11, or AAV12.

some embodiments, the AAV is AAV1, AAV2, AAV3, AAV4, AAV5, AAV6, AAV7, AAV8, AAV9, AAVrh8, AAVrh8R, AAV10, AAVrh10, AAV11, or AAV12.

In some embodiments, the AAV is AAV2 or AAV8.

In some embodiments, the AAV particle comprises a DNA nucleic acid encoding an STC-2 fragment (or variant thereof) or encoding a modified protein comprising an STC-2 fragment (or variant thereof). In some embodiments, the DNA nucleic acid encodes a peptide growth hormone receptor antagonist or a fragment thereof. In some embodiments, the DNA nucleic acid encodes a growth hormone releasing hormone antagonists or a fragment thereof. In some embodiments, the DNA nucleic acid encodes a peptide inhibitor of PAPP-A or a fragment thereof.

AAVs are increasingly used for gene delivery in basic scientific research and therapeutic applications because of their ability to transduce both dividing and non-dividing cells, their long-term persistence as episomal DNA in infected cells, and their low immunogenicity. These characteristics make them appealing for applications in gene therapy, including according to the methods of the present disclosure.

Methods of producing and packaging AAVs is well known in the art. In some embodiments, plasmid vectors are transfected into mammalian cells (e.g., HEK293 cells) using standard transfection protocols. An AAV may comprise a plasmid that contains a transgene cassette (e.g., containing a nucleic acid which encodes an STC-2 fragment, or variant thereof, or which encodes a modified protein comprising an STC-2 fragment, or variant thereof) flanked by inverted terminal repeat (ITR) sequences from a parental AAV virus. The transgene cassette has a promoter sequence and that drives transcription of a heterologous nucleic acid in the nucleus of a target cell.

Modified Proteins

Aspects of the present disclosure include methods for increasing lifespan in a non-rodent mammal in need thereof or in a mature mammal in need thereof. The methods can comprise administering to the mammal a therapeutically effective amount of a composition including a modified protein comprising a target protein or a functional fragment or variant thereof (e.g., a fragment of STC-2) and a modification that improves the half-life of the target protein or the functional fragment or variant thereof in serum. In some embodiments, the target protein is a peptide growth hormone receptor antagonist or a fragment thereof. In some embodiments, the target protein is a growth hormone releasing hormone antagonists or a fragment thereof. In some embodiments, the target protein is a peptide inhibitor of PAPP-A or a fragment thereof.

Additionally, methods for increasing lifespan in a non-rodent mammal in need thereof or in a mature mammal in need thereof comprise administering a nucleic acid encoding a modified protein including a target protein or a functional fragment or variant thereof (e.g., a fragment of STC-2) and a modification that improves the half-life of the target protein or the functional fragment or variant thereof in serum. In some embodiments, the target protein is a peptide growth hormone receptor antagonist or a fragment thereof. In some embodiments, the target protein is a growth hormone releasing hormone antagonists or a fragment thereof. In some embodiments, the target protein is a peptide inhibitor of PAPP-A or a fragment thereof.

In some embodiments, the modification that improves the half-life of the target protein or the functional fragment or variant thereof in serum is derived from an Fc domain of an antibody, a transferrin, an albumin, a CTP sequence, an XTEN sequence, an ELP sequence, a PAS sequence, a HAP sequence, and a GLK sequences, polyethylene glycol (PEG), or a combination thereof. When the modification is a peptide or a polypeptide, the modified protein can be considered a fusion protein. When the modification is PEG, the modified protein can be considered a PEGylated protein.

In some embodiments, the modification that improves the half-life of the target protein or the functional fragment or variant thereof in serum comprises an Fc domain of an antibody (e.g., of IgG, IgA, IgD, and IgE, inclusive of subclasses (e.g., IgG1, IgG2, IgG3, and IgG4, and IgA1 and IgA2).

In some embodiments, the Fc domain of the antibody contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In some embodiments, the amino acid substitution at amino acid residue 250 is a substitution with glutamine. In some embodiments, the amino acid substitution at amino acid residue 252 is a substitution with tyrosine, phenylalanine, tryptophan or threonine. In some embodiments, the amino acid substitution at amino acid residue 254 is a substitution with threonine. In some embodiments, the amino acid substitution at amino acid residue 256 is a substitution with serine, arginine, glutamine, glutamic acid, aspartic acid, or threonine. In some embodiments, the amino acid substitution at amino acid residue 308 is a substitution with threonine. In some embodiments, the amino acid substitution at amino acid residue 309 is a substitution with proline. In some embodiments, the amino acid substitution at amino acid residue 311 is a substitution with serine. In some embodiments, the amino acid substitution at amino acid residue 385 is a substitution with arginine, aspartic acid, serine, threonine, histidine, lysine, alanine or glycine. In some embodiments, the amino acid substitution at amino acid residue 386 is a substitution with threonine, proline, aspartic acid, serine, lysine, arginine, isoleucine, or methionine. In some embodiments, the amino acid substitution at amino acid residue 387 is a substitution with arginine, proline, histidine, serine, threonine, or alanine. In some embodiments, the amino acid substitution at amino acid residue 389 is a substitution with proline, serine or asparagine. In some embodiments, the amino acid substitution at amino acid residue 416 is a substitution with serine. In some embodiments, the amino acid substitution at amino acid residue 428 is a substitution with leucine. In some embodiments, the amino acid substitution at amino acid residue 433 is a substitution with arginine, serine, isoleucine, proline, or glutamine. In some embodiments, the amino acid substitution at amino acid residue 434 is a substitution with histidine, phenylalanine, or tyrosine.

In some embodiments, the Fc domain of the antibody comprises an IgG constant region, which comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In some embodiments, the IgG constant region includes a triple M252Y/S254T/T256E mutation or YTE mutation. In an embodiment, the IgG constant region includes a triple H433K/N434F/Y436H mutation or KFH mutation. In some embodiments, the IgG constant region includes an YTE and KFH mutation in combination.

In some embodiments, the modification that improves the half-life of the target protein or the functional fragment or variant thereof in serum comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, 1307A, E380A, 1253A, H310A, M428L, H433K, N434A, N434F, N434S, and H435A. In some embodiments, the IgG constant region comprises a M428L/N434S mutation or LS mutation. In an embodiment, the IgG constant region comprises a T250Q/M428L mutation or QL mutation. In an embodiment, the IgG constant region comprises an N434A mutation. In an embodiment, the IgG constant region comprises a T307A/E380A/N434A mutation or AAA mutation. In an embodiment, the IgG constant region comprises an 1253A/H310A/H435A mutation or IHH mutation. In an embodiment, the IgG constant region comprises a H433K/N434F mutation. In an embodiment, the IgG constant region comprises a M252Y/S254T/T256E and a H433K/N434F mutation in combination.

In some embodiments, the modification that improves the half-life of the target protein or the functional fragment or variant thereof in serum is derived from an Fc domain of an IgG4 and comprises one or more mutations selected from S228P, T250Q, M428L, V308T, L309P, and Q311S in accordance with Kabat numbering. In some embodiments, the domain comprises 1, or 2, or 3, or 4, or 5 of these mutations.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al, Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

Any of the herein-disclosed mutations in an Fc domain of an antibody or in an IgG constant region can be combined into a modification that improves the half-life of the target protein or the functional fragment or variant thereof in serum.

The modification that improves the half-life of the target protein or the functional fragment or variant thereof in serum may comprise transferrin, e.g., human transferrin. Use of transferrin in a modified protein is described in, at least, Kim, et al., J Pharmacol Exp Ther. 2010 September; 334(3): 682-692; the contents of which is incorporated by reference in its entirety.

In some embodiments, the modification that improves the half-life of the functional fragment or variant thereof in serum in serum may comprise albumin, e.g., human serum albumin (HSA). Use of HSA in a modified protein is described in, at least, Rogers, et al., Curr Pharm Des. 2015; 21(14):1899-907; the contents of which is incorporated by reference in its entirety.

In some embodiments, the modification that improves the half-life of the functional fragment or variant thereof in serum may comprise a CTP sequence. In some embodiments, the CTP sequence comprises a thirty-one amino-acid-residue CTP consisting of the sequence FQSSSS*KAPPPS*LPSPS*RLPGPS*DTPILPQ (SEQ ID NO: 16). Use of a CTP sequence in a modified protein is described in, at least, Birken and Canfield J Biol Chem. 1977; 252:5386-5392 and Fares et al., Proc Natl Acad Sci USA. 1992; 89:4304-4308; the contents of each of which is incorporated by reference in its entirety.

In some embodiments, the modification that improves the half-life of the functional fragment or variant thereof in serum may comprise an “XTEN” sequence. Use of XTEN sequences in a modified protein is described in, at least, Haeckel, et al., PLoS One. 2016; 11(6): e0157193; the contents of which is incorporated by reference in its entirety.

In some embodiments, the modification that improves the half-life of the functional fragment or variant thereof fragment in serum may comprise polyethylene glycol (PEG). Use of PEG in a modified protein is described in, at least, Damodaran and Fee European Pharmaceutical Review, 2010, Issue 1; Fee and Damodaran, 2012, Chapter 7 “Production of PEGylated Proteins” in Biopharmaceutical Production Technology; and Awwad et al., 2018, Chapter 2, “The case for protein PEGylation” in Engineering of Biomaterials for Drug Delivery Systems; the contents of each of which is incorporated by reference in its entirety.

In some embodiments, the modifications that improve the half-life of the modified proteins in serum are described, at least, in Strohl W R “Modified proteins for Half-Life Extension of Biologics as a Strategy to Make Biobetters.” BioDrugs. 2015 August; 29(4):215-39; the contents of which are incorporated by reference in its entirety.

In some embodiments, the nucleic acid encoding a modified protein further encodes a linker located between the functional fragment or variant thereof fragment and the modification that improves the half-life of the functional fragment or variant thereof fragment in serum. Additionally, a modified protein of the present disclosure (e.g., an in vitro translated modified protein) further comprises a linker located between the functional fragment or variant thereof fragment and the modification that improves the half-life of the functional fragment or variant thereof fragment in serum.

When a modified protein comprises a linker, the linker provides further distance between the functional fragment or variant thereof fragment and the modification that improves the half-life of the functional fragment or variant thereof fragment in serum. Without wishing to be bound by theory, this distance may help avoid steric hindrance that would prevent the normal functions of the functional fragment or variant thereof fragment (e.g., binding to its respective binding partners). In the embodiments where the functional fragment or variant thereof fragment is STC-2, the distance may help avoid the hindrance that prevents the STC-2 portion of the modified protein from binding pregnancy-associated plasma protein-A (PAPP-A) and/or PAPP-A2. In some embodiments, the linker is a polypeptide linker comprising between about 2 amino acids and about 500 amino acids, or is a synthetic linker, e.g., comprising PEG.

The modified protein may be in vitro translated. As such the protein is synthesized from a recombinant cell. For example, a cell that has been transfected or transformed with a nucleic acid that expresses an mRNA encoding the modified protein. However, an in vitro translated modified protein may have been synthesized chemically, e.g., via direct synthesis methods. Methods for producing in vitro translated proteins are well-known in the art. When a functional fragment or variant thereof fragment or a modified protein comprising the functional fragment or variant thereof fragment is administered to a mammal, it us understood that the functional fragment or variant thereof fragment or the modified protein comprising the functional fragment or variant thereof fragment may have been in vitro translated.

In some embodiments where the modified protein is provided as an in vitro translated protein, the modified protein is incapable of crossing the blood brain barrier.

Biomarkers

Methods of the present disclosure may further comprise detecting levels of biomarkers in blood samples, e.g., a serum sample.

The method may further comprise detecting an amount of one or more biomarkers in a first blood sample, wherein the first blood sample is obtained from the mammal before administering the composition.

The method may further comprise detecting an amount of the one or more biomarkers in a second blood sample, wherein the second blood sample is obtained from the mammal after administering the composition.

The method may further comprise detecting an amount of the one or more biomarkers in a third (or subsequent) blood sample, wherein the third (or subsequent) blood sample is obtained from the mammal after administering the composition at least twice.

The method may further comprise detecting an amount of one or more biomarkers in a first blood sample, wherein the first blood sample is obtained from the mammal before administering the composition, and detecting an amount of the one or more biomarkers in a second blood sample, wherein the second blood sample is obtained from the mammal after administering the composition. In this embodiment, the amount of the one or more biomarkers in the first sample is compared to the amount of the one or more biomarkers in the second sample.

The method may further comprise detecting an amount of one or more biomarkers in a first blood sample or in a second blood sample and detecting an amount of the one or more biomarkers in a third (or subsequent) blood sample, wherein the third (or subsequent) blood sample is obtained from the mammal after administering the composition at least twice. In this embodiment, the amount of the one or more biomarkers in the first sample or the second sample is compared to the amount of the one or more biomarkers in the third (or subsequent) blood sample.

In some embodiments, the amount of the one or more biomarkers in a sample is compared to a historical control.

The therapeutically effective amount of the composition may produce an increase in the amount of one or more biomarkers selected from phosphorylated IGF-1R, glucose, insulin, non-esterified fatty acids (NEFA), cholesterol, thyroid hormones, GH-receptor, phosphorylated GH-receptor, insulin, IGFBP1, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, and Rac, mechanistic target of rapamycin (mTOR), AMP-activated protein kinase (AMPK), c-reactive protein (CRP), IL-6, TNFα-receptor I or II, IL-8, IL-2, interferon gamma, adiponectin, FGF-21, FGF-23, GDF15, Cystatin C, NT-proBNP, hemoglobin A1c, FSTL3, total homocysteine (tHcy), alpha Klotho, beta Klotho, Glucose transporter type 4 (GLUT4), Serum alkaline phosphatase (AP), serum aspartate aminotransferase (AST), serum alanine aminotransferase (ALT), serum albumin, serum triglycerides, serum free fatty acids, Dehydroepiandrosterone sulfate (DHEAS), NT-proBNP, Follistatin-like 3 FSTL3, myostatin (GDF8), Transforming growth factor beta (TGF-β), or a homolog thereof in the second sample relative to the first sample and/or relative to a historical control.

In some embodiments where the composition comprises a fragment of STC-2, the therapeutically effective amount of the composition may produce an increase in the amount of one or more biomarkers selected from ApoA1 isoform 1, ApoA1 isoform 2, ApoA4, haptoglobin isoform 2, haptoglobin isoform 3, immunoglobulin kappa chain isoform 1, immunoglobulin kappa chain isoform 2, immunoglobulin kappa chain isoform 3, transthyretin isoform 1, transthyretin isoform 2, α2-macroglobulin isoform 1, and α2-macroglobulin isoform 2, or a homolog thereof in the second sample relative to the first sample and/or relative to a historical control.

The therapeutically effective amount of the composition may produce a decrease in the amount of one or more biomarkers selected from phosphorylated IGF-1R, glucose, insulin, non-esterified fatty acids (NEFA), cholesterol, thyroid hormones, GH-receptor, phosphorylated GH-receptor, insulin, IGFBP1, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, and Rac, mechanistic target of rapamycin (mTOR), AMP-activated protein kinase (AMPK), c-reactive protein (CRP), IL-6, TNFα-receptor I or II, IL-8, IL-2, interferon gamma, adiponectin, FGF-21, FGF-23, GDF15, Cystatin C, NT-proBNP, hemoglobin A1c, FSTL3, total homocysteine (tHcy), alpha Klotho, beta Klotho, Glucose transporter type 4 (GLUT4), Serum alkaline phosphatase (AP), serum aspartate aminotransferase (AST), serum alanine aminotransferase (ALT), serum albumin, serum triglycerides, serum free fatty acids, Dehydroepiandrosterone sulfate (DHEAS), NT-proBNP, Follistatin-like 3 FSTL3, myostatin (GDF8), Transforming growth factor beta (TGF-(3), or a homolog thereof in the second sample relative to the first sample and/or relative to a historical control.

In some embodiments where the composition comprises a fragment of STC-2, the therapeutically effective amount of the composition may produce a decrease in the amount of one or more biomarkers selected from ApoA1 isoform 1, ApoA1 isoform 2, ApoA4, haptoglobin isoform 2, haptoglobin isoform 3, immunoglobulin kappa chain isoform 1, immunoglobulin kappa chain isoform 2, immunoglobulin kappa chain isoform 3, transthyretin isoform 1, transthyretin isoform 2, α2-macroglobulin isoform 1, and α2-macroglobulin isoform 2, or a homolog thereof in the second sample relative to the first sample and/or relative to a historical control.

In some embodiments, a herein described composition comprises peptide growth hormone receptor antagonists, growth hormone releasing hormone antagonists, and/or additional peptide inhibitors of PAPP-A. In some embodiments, a herein described composition comprises peptide growth hormone receptor antagonists, growth hormone releasing hormone antagonists, and/or additional peptide inhibitors of PAPP-A, or nucleic acids encoding the same. A therapeutically effective amount of the composition may produce a decrease in the amount of one or more biomarkers selected from ApoA1 isoform 1, ApoA1 isoform 2, ApoA4, haptoglobin isoform 2, haptoglobin isoform 3, immunoglobulin kappa chain isoform 1, immunoglobulin kappa chain isoform 2, immunoglobulin kappa chain isoform 3, transthyretin isoform 1, transthyretin isoform 2, α2-macroglobulin isoform 1, and α2-macroglobulin isoform 2, or a homolog thereof in the second sample relative to the first sample and/or relative to a historical control.

The therapeutically effective amount of a herein described composition may produce a change in the amount of one or more biomarkers selected from phosphorylated IGF-1R, glucose, insulin, non-esterified fatty acids (NEFA), cholesterol, thyroid hormones, GH-receptor, phosphorylated GH-receptor, insulin, IGFBP1, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, and Rac, mechanistic target of rapamycin (mTOR), AMP-activated protein kinase (AMPK), c-reactive protein (CRP), IL-6, TNFα-receptor I or II, IL-8, IL-2, interferon gamma, adiponectin, FGF-21, FGF-23, GDF15, Cystatin C, NT-proBNP, hemoglobin Alc, FSTL3, total homocysteine (tHcy), alpha Klotho, beta Klotho, Glucose transporter type 4 (GLUT4), Serum alkaline phosphatase (AP), serum aspartate aminotransferase (AST), serum alanine aminotransferase (ALT), serum albumin, serum triglycerides, serum free fatty acids, Dehydroepiandrosterone sulfate (DHEAS), NT-proBNP, Follistatin-like 3 FSTL3, myostatin (GDF8), Transforming growth factor beta (TGF-β) in the second sample relative to the first sample and/or relative to a historical control.

In some embodiments where the composition comprises a fragment of STC-2, the therapeutically effective amount of the composition may produce a change in the amount of one or more biomarkers selected from IGF-1, IGF-2, IGF-1R, phosphorylated IGF-1R, Growth Hormone (GH), GH-receptor, phosphorylated GH-receptor, IGFBP3, IGFBP3-IGF-1 isoform levels, pregnancy-associated plasma protein A (PAPP-A), PAPP-A2, P13Kinase, IRS-1, Ras/Raf, MEK, ERK, Akt, and Rac, or a homolog thereof in the second sample relative to the first sample and/or relative to a historical control.

In some embodiments where the composition comprises a fragment of STC-2, the therapeutically effective amount of the composition may produce a change (e.g., decrease) in the amount of free IGF-1 to IGF-1 that is bound to an insulin-like growth factor-binding protein (IGFBP, e.g., one of IGFBP1 to IGFBP6) in the second sample relative to the first sample and/or relative to a historical control. In some embodiments where the composition comprises a fragment of STC-2, the therapeutically effective amount of the composition may produce a change (e.g., decrease) the amount of cleaved insulin-like growth factor-binding protein (IGFBP, e.g., one of IGFBP1 to IGFBP6) in the second sample relative to the first sample and/or relative to a historical control. In some cases, the IGFBP is IGFBP-4 or is IGFBP-5. In some embodiments where the composition comprises a fragment of STC-2, the therapeutically effective amount of the composition may produce a change (e.g., decrease) the enzymatic activity of pregnancy-associated plasma protein A (PAPP-A) and/or PAPP-A2 in the second sample relative to the first sample and/or relative to a historical control.

The therapeutically effective amount of the composition may produce a change in other physiological markers selected from prostate-specific antigen (PSA), c-reactive protein (CRP), IL-6, Serum alkaline phosphatase (AP), serum aspartate aminotransferase (AST), serum alanine aminotransferase (ALT), insulin, serum glucose, serum albumin, serum lipids, hemoglobin, hematocrit, platelet count, serum electrolytes, hepatic enzymes, estimated glomerular filtration rates, thymic fat-free fraction (TFFF), bone marrow fat-free fraction (BMFFF), total monocytes, lymphocyte-to-monocyte ratio, lymphocyte-to-CD38+monocyte ratio, CD38-positive monocytes, PD-1-positive CD8 cells, normalized naïve CD8 cells, normalized naïve CD4 cells, percentage of CD31+CD45RA+CD4+ cells, or serum FGF-21 in the second sample relative to the first sample and/or relative to a historical control.

The therapeutically effective amount of the composition may produce a change in an epigenetic marker in the second sample relative to the first sample and/or relative to a historical control. In some embodiments, the epigenetic marker is an amount of methylated DNA, e.g., which is characterized via one or more of the Horvath epigenetic method, PhenoAge method, Hannum epigenetic method, or GrimAge method. For further descriptions of epigenetic aging methods, see, e.g., Horvath Genome Biology, 2013, 14(10):R11 and Ecker and Beck Aging, 2019, 11(2): 833-835 [and the references cited therein, including Horvath et al., Aging, 2018, 10 (7): 1758-1775; Levine et al., Aging, 2018, 10(4):573-591; Lu et al., Aging, 2019, 11(2):303-327; and Hannum et al., Mol Cell., 2013, 49(2):359-367], the contents of each of which is incorporated by reference in its entirety.

In some embodiments, the epigenetic marker is one or more of DNA sequences covalently modified by cytosine methylation and hydroxymethylation or of histone proteins by lysine acetylation, lysine and arginine methylation, serine and threonine phosphorylation, and lysine ubiquitination, or sumoylation.

As used herein, a change in a biomarker may be an increase in the biomarker in a second sample obtained from a mammal relative to a first sample or relative to a historical control. The terms increased or increase, and the like, are used herein to generally mean an increase by a measurable amount, e.g., a statistically significant amount. In some embodiments, the terms increased or increase means an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase, or a greater increase, as compared to a reference level, e.g., an earlier-collected sample from a subject (e.g., before administration of a composition of the present disclosure), standard, or control, including historical control. Other examples of increase include an increase of at least 2-fold, at least 5-fold, at least 10-fold, at least 20-fold, at least 50-fold, at least 100-fold, at least 1000-fold or as compared to a reference level, e.g., an earlier-collected sample from a subject (e.g., before administration of a composition of the present disclosure), standard, or control, including historical control.

Alternately, a change in a biomarker may be a decrease in the biomarker in a second sample obtained from a mammal relative to a first sample or relative to a historical control. The terms decreased, or decrease, and the like, are used herein generally to mean a decrease by a measurable amount, e.g., a statistically significant amount. In some embodiments, decreased or decrease means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, as compared to a reference level, e.g., an earlier-collected sample from a subject (e.g., before administration of a composition of the present disclosure), standard, or control, including historical control.

As used herein, a historical control is where previous-obtained data is used to compare with new data. Information, e.g., the standard, median, normal, or pre-treatment serum amount/level/concentration of a biomarker, is essentially borrowed from historical data. The historical data is usually from a subject with the same characteristics, e.g., species, strain, breed, sex, age, weight, size, health, and/or disease status.

Subjects

In some embodiments, the subject is a mammal, e.g., a human, mouse, rat, guinea pig, dog, cat, horse, cow, pig, rabbit, sheep, or non-human primate, such as a monkey, chimpanzee, or baboon. In some embodiments, the mammal is a non-rodent. In some embodiments, the mammal is a dog. In some embodiments, the mammal is a human.

In some embodiments, the mammal has reached maturity. As used herein, the term mature or maturity, and the like, refers to a mammal that is capable of sexual reproduction and/or a mammal that has achieved its adult height and/or length.

In some embodiments, the mammal is administered a herein-disclosed composition as it is nearing or once it has reached halfway to its expected lifespan for the mammal's species, strain, breed, sex, and/or age. It is known that small dog breeds (e.g., Chihuahua) have longer expected lifespans than larger dog breeds (e.g., Great Dane). Accordingly, a Chihuahua, which has an expected lifespan of 15 years, will reach halfway to its expected lifespan at about 7 years (or earlier); thus, a Chihuahua may be administered a composition beginning around 7 years of age. On the other hand, a Great Dane, which has an expected lifespan of 7 years, will halfway to its expected lifespan at about 3 years; thus, a Great Dane may be administered a composition beginning around 3 years of age (or earlier). As disclosed herein, a mammal may be administered a composition once it has reached maturity; thus, either dog breed may be administered a composition about its first-year birthday.

Any dog breed can be administered a composition of the present disclosure and treated by a herein-described method. Lists of the most common dog breeds, identified by year, are maintained by the American Kennel Club. See, the World Wide Web (www) at akc.org/expert-advice/news/most-popular-dog-breeds-full-ranking-list; the lists of dog breeds, published at the time of the present application's filing, are incorporated by reference in their entireties. Illustrative common dog breeds include Retrievers (Labrador), German Shepherd Dogs, Retrievers (Golden), French Bulldogs, Bulldogs, Beagles, Poodles, Rottweilers, Pointers (German Shorthaired), Yorkshire Terriers, Boxers, Dachshunds, Pembroke Welsh Corgis, Siberian Huskies, Australian Shepherds, Great Danes, Doberman Pinschers, Cavalier King Charles Spaniels, Miniature Schnauzers, Shih Tzu, Boston Terriers, Bernese Mountain Dogs, Pomeranians, Havanese, Shetland Sheepdogs, Brittanys, Spaniels (English Springer), Pugs, Mastiffs, Spaniels (Cocker), Vizslas, Cane Corso, Chihuahuas, Miniature American Shepherds, Border Collies, Weimaraners, Maltese, Collies, Basset Hounds, and Newfoundlands.

In some embodiments, the human is an adult human. In some embodiments, the human has an age in a range of from about 10 to about 15 years old, from about 15 to about 20 years old, from about to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old, or older

In some embodiments, the subject is a non-human animal, and therefore the disclosure pertains to veterinary use. In some embodiments, the non-human animal is a household pet, e.g., a dog. In some embodiments, the non-human animal is a livestock animal.

The herein-disclosed compositions and methods treat, prevent, reduce the severity of, and/or delay the onset of various aging-associated conditions, e.g., chronic diseases and disabilities/conditions of aging. Illustrative aging-associated conditions include age-related macular degeneration (AMD), Alzheimer's disease, arthritis, atherosclerosis and cardiovascular disease, benign prostatic hyperplasia (BPH), bone atrophy, cancer, cataracts, constipation, decrease in visual acuity, decrease in overall energy, delirium, dementia, depression, diminished peripheral vision, greater risk of heat stroke or hypothermia, hearing loss, hypertension, increased susceptibility to infection (including influenza and pneumonia), memory loss, metabolic syndrome, muscle atrophy, osteoporosis, reduced metabolism (including increased risk for obesity), reduced reflexes and coordination including difficulty with balance, respiratory disease, shingles, type 2 diabetes, urologic changes (including incontinence), whitening or graying of hair, and wrinkling and sagging skin (including loss of skin elasticity). Aged non-human subjects experience similar, homologous, and/or equivalent aging-associated conditions.

As used herein, the term healthspan refers to the part of a subject's life during which they are generally in good health or a period of life spent in good health, free from chronic diseases and disabilities/conditions of aging. Accordingly, a herein-disclosed composition or method that treats, prevents, reduces the severity of, and/or delay the onset of various aging-associated conditions, as mentioned above, improves the healthspan of a mammal. In some embodiments, the chronic disease is a cancer; thus, improving the healthspan of the mammal comprises treating a cancer and/or delaying or preventing the onset of the cancer in the mammal.

Compositions and Administrations

An aspect of the present disclosure is a composition comprising a therapeutically effective amount of nucleic acid (e.g., encoding an STC-2 fragment or a modified protein comprising the same) or a modified protein (e.g., comprising an STC-2 fragment) for increasing lifespan of a mammal. In some embodiments, the composition is a lipid nanoparticle composition. In some embodiments, the composition is a liposome composition. In some embodiments, the nucleic acid encodes a peptide growth hormone receptor antagonist or a fragment thereof. In some embodiments, the nucleic acid encodes a growth hormone releasing hormone antagonists or a fragment thereof. In some embodiments, the nucleic acid encodes a peptide inhibitor of PAPP-A or a fragment thereof.

The term therapeutically effective amount is meant the amount of a composition, nucleic acid, modified protein, and/or viral particle which can provide a desired therapeutic benefit, e.g., increasing lifespan, promoting longevity, and/or preventing, reducing the severity of, or delaying the onset of various aging-associated conditions.

In some embodiments, increasing lifespan comprises an at least 5% increase in lifespan relative to the expected or median lifespan of a mammal of similar species, strain, or breed, e.g., an at least 10%, at least 15%, at least 20%, or at least 25% increase in lifespan.

In a composition, the nucleic acid or modified protein may be packaged in a liposome or lipid nanoparticle. In a composition, the nucleic acid may be included in an expression vector (e.g., a viral expression vector). In this aspect, the composition for use in any herein-disclosed method for increasing lifespan of a mammal may also increase the healthspan of the mammal Increasing healthspan may comprise treating a cancer and/or delaying or preventing the onset of the cancer in the mammal.

The compositions of the present disclosure are formulated to be suitable for in vivo administration to a mammal. Such compositions can optionally comprise a suitable amount of a pharmaceutically acceptable excipient so as to provide the form for proper administration. Pharmaceutical excipients can be liquids, such as water or saline. In addition, auxiliary, stabilizing, thickening, lubricating, and coloring agents can be used. The pharmaceutically acceptable excipients are sterile when administered to a subject. Water is a useful excipient when any composition described herein is administered intravenously. In some embodiments, the compositions described herein are suspended in a saline buffer (including, without limitation Ringer's, TBS, PBS, HEPES, HBSS, and the like). Saline solutions and aqueous dextrose and glycerol solutions can also be employed as liquid excipients, specifically for injectable solutions. Suitable pharmaceutical excipients also include starch, glucose, lactose, sucrose, glycerol monostearate, sodium chloride, dried skim milk, glycerol, propylene, glycol, water, ethanol and the like. Any composition described herein, if desired, can also comprise pH buffering agents.

Dosage forms suitable for parenteral administration (e.g., intravenous injection or infusion, intraarterial injection or infusion, intramuscular injection, intraperitoneal injection, subcutaneous injection, and intra-arterial injection or infusion) include, for example, solutions, suspensions, dispersions, emulsions, and the like.

The dosage of any herein-disclosed composition can depend on several factors including the characteristics of the mammal to be administered. Examples of characteristics include species, strain, breed, sex, age, weight, size, health, and/or disease status. Moreover, the dosage may depend on whether the administration is the first time the subject received a composition of the present disclosure or if the subject has previously received a composition of the present disclosure. Additionally, pharmacogenomic (the effect of genotype on the pharmacokinetic, pharmacodynamic or efficacy profile of a composition) information about a particular subject may affect dosage used. Furthermore, the exact individual dosages can be adjusted somewhat depending on a variety of factors, including the specific composition being administered, the time of administration, the route of administration, the nature of the formulation, and the rate of excretion. Some variations in the dosage can be expected.

Moreover, the dosage depends on the specific composition administered, e.g., comprising a nucleic acid or modified protein (within a liposome or nanoparticle or not within these) or a viral expression vector.

The effective dose of a nucleic acid encoding a target protein or a functional fragment or variant thereof (e.g., encoding an STC-2 fragment or variant thereof) or encoding a modified protein comprising a target protein or a functional fragment or variant thereof (e.g., an STC-2 fragment or variant thereof), in a liposome or lipid nanoparticle formulation or as a naked RNA, may be about 100 ng to about 2000 ng, or about 200 ng to about 1900 ng, or about 300 ng to about 1800 ng, or about 400 ng to about 1700 ng, or about 500 ng to about 1600 ng, or about 600 ng to about 1500 ng, or about 700 ng to about 1400 ng, or about 800 ng to about 1300 ng, or about 900 ng to about 1200 ng, or about 1000 ng to about 1 100 ng, or about 500 ng to about 2000 ng, or about 500 ng to about 1500 ng, or about 500 ng to about 1000 ng, or about 1000 ng to about 1500 ng, or about 1000 ng to about 2000 ng, or about 1500 ng to about 2000 ng, or about 100 ng to about 500 ng, or about 200 ng to about 400 ng, or about ng to about 100 ng, or about 20 ng to about 90 ng, or about 30 ng to about 80 ng, or about 40 ng to about 70 ng, or about 50 ng to about 60 ng.

The effective dose of a nucleic acid encoding a target protein or a functional fragment or variant thereof (e.g., encoding an STC-2 fragment or variant thereof) or encoding a modified protein comprising a target protein or a functional fragment or variant thereof (e.g., an STC-2 fragment or variant thereof), in a liposome or lipid nanoparticle formulation or as a naked RNA, may be no more than about 50 ng, or about 100 ng, or about 200 ng, or about 300 ng, or about 400 ng, or about 500 ng, or about 600 ng, or about 700 ng, or about 800 ng, or about 900 ng, or about 1000 ng, or about 1 100 ng, or about 1200 ng, or about 1300 ng, or about 1400 ng, or about 1500 ng, or about 1600 ng, or about 1700 ng, or about 1800 ng, or about 1900 ng, or about 2000 ng, or about 3000 ng, or about 4000 ng, or about 5000 ng.

The effective dose of a nucleic acid encoding a target protein or a functional fragment or variant thereof (e.g., encoding an STC-2 fragment or variant thereof) or encoding a modified protein comprising a target protein or a functional fragment or variant thereof (e.g., an STC-2 fragment or variant thereof), in a liposome or lipid nanoparticle formulation or as a naked RNA, may be about 50 ng, or about 100 ng, or about 200 ng, or about 300 ng, or about 400 ng, or about 500 ng, or about 600 ng, or about 700 ng, or about 800 ng, or about 900 ng, or about 1000 ng, or about 1 100 ng, or about 1200 ng, or about 1300 ng, or about 1400 ng, or about 1500 ng, or about 1600 ng, or about 1700 ng, or about 1800 ng, or about 1900 ng, or about 2000 ng, or about 3000 ng, or about 4000 ng, or about 5000 ng.

The dosage of a herein-disclosed modified protein (e.g., an in vitro translated modified protein) may be about 0.1 mg to about 250 mg per administration, about 1 mg to about 20 mg per administration, or about 3 mg to about 5 mg per administration. Generally, the dosage of these compositions may be about 0.1 mg to about 1500 mg per administration, or about 0.5 mg to about 10 mg per administration, or about 0.5 mg to about 5 mg per administration, or about 200 to about 1,200 mg per administration (e.g., about 200 mg, about 300 mg, about 400 mg, about 500 mg, about 600 mg, about 700 mg, about 800 mg, about 900 mg, about 1,000 mg, about 1,100 mg, about 1,200 mg per administration). The dosage of a herein-disclosed modified protein may be greater than 1,500 mg per administration, e.g., 2 g, 3 g, 4 g, 5 g, 6 g, 7 g, 8 g, 9 g, 10 g, or more per administration.

In some embodiments, the composition comprises a viral expression vector. The viral genome (vg) concentration of the composition that is administered may be between 1.0×10¹¹ vg per kilogram (kg) and 1.0×10¹⁶ vg/kg. In some embodiments, the concentration of infectious particles is at least or about 10⁷, 10⁸, 10⁹, 10¹⁰, 10¹¹, 10¹², 10¹³, 10¹⁴, 10¹⁵, 10¹⁶, or 10¹⁷. In some embodiments, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some embodiments, the concentration of the infectious particles 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some embodiments, the concentration of the infectious particles 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some embodiments, the concentration of the infectious particles 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some embodiments, the concentration of the infectious particles 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some embodiments, the concentration of the infectious particles 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some embodiments, the concentration of the infectious particles 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some embodiments, the concentration of the infectious particles 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

When a composition comprises a viral particle, the administering may be performed once. Alternatively, the administering occurs at least twice. When a second composition is administered, the second composition may be administered a year or longer after the first administration, e.g., five years after the first administration. In some embodiments, the administering occurs at least three, at least four, at least five, at least six, at least seven, at least eight, at least nine, at least ten, or more additional times.

When the composition comprises a nucleic acid, the administering may be performed once. Alternatively, the administering occurs at least twice. When a second composition is administered, the second composition may be administered an hour or hours, a day, a week, a month, a year or longer after the first administration. In some embodiments, the administering occurs at least three, at least four, at least five, at least six, at least seven, at least eight, at least nine, at least ten, or more additional times. When a subsequent composition is administered, the subsequent composition may be administered an hour or hours, a day, a week, a month, a year or longer after the immediately previous administration.

When the composition comprises a modified protein, the administering may be performed once. Alternatively, the administering occurs at least twice. When a second composition is administered, the second composition may be administered an hour or hours, a day, a week, a month, a year or longer after the first administration. In some embodiments, the administering occurs at least three, at least four, at least five, at least six, at least seven, at least eight, at least nine, at least ten, or more additional times. When a subsequent composition is administered, the subsequent composition may be administered an hour or hours, a day, a week, a month, a year or longer after the immediately previous administration.

A subsequent administration of a composition may be 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, 5 years, or 10 years after the immediately previous administration. The effective dosage ranges may be adjusted based on a subject's response to a previous administration and/or the length of time between administrations.

A subject may be administered a composition of one type (e.g., comprising a nucleic acid) and is later administered a composition of a second type (e.g., comprising a modified protein). Any herein-disclosed compositions type, i.e., comprising a nucleic acid (within a liposome or lipid nanoparticle or not within these), comprising a modified protein (within a liposome or lipid nanoparticle or not within these), or comprising a viral expression vector, may be included in a first administration and any herein-disclosed composition type (the same or different) may be included in a subsequent administration.

Methods for Increasing Healthspan

Administering the composition may further improve the healthspan of the mammal. As used herein, the term healthspan refers to the part of a subject's life during which they are generally in good health or a period of life spent relatively free from chronic diseases and disabilities/conditions of aging.

In some embodiments, the chronic disease is a cancer. Cancer refers to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. Tumor cells that have metastasized may be like those in the original tumor. For example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

In some embodiments, improving the healthspan of the mammal comprises treating a cancer and/or delaying or preventing the onset of the cancer in the mammal Thus, a therapeutically-effective amount of a composition is capable of treating a cancer and/or preventing or delaying the onset of the cancer in the mammal.

Representative cancers include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments where the target protein is STC-2, the cancer comprises a cell that expresses an insulin like growth factor receptor.

In some embodiments where the target protein is STC-2, the cancer comprises a cell that expresses pregnancy-associated plasma protein-A (PAPP-A) and/or PAPP-A2.

### Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

By preventing is meant, at least, avoiding the occurrence of a disease and/or reducing the likelihood of acquiring the disease. By treating is meant, at least, ameliorating or avoiding the effects of a disease, including reducing a sign or symptom of the disease.

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

Reference herein to “one embodiment,” “one version,” or “one aspect” can include one or more such embodiments, versions or aspects, unless otherwise clear from the context.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

The term “nucleic acid” as used herein refers to a polymer containing at least two deoxyribonucleotides or ribonucleotides in either single- or double-stranded form and includes DNA and RNA. DNA can be in the form of, e.g., antisense molecules, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA can be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically stated otherwise, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

“Nucleotides” contain a sugar deoxyribose (DNA) or ribose (RNA), a base, and a phosphate group. Nucleotides are linked together through the phosphate groups.

The term “gene” refers to a nucleic acid (e.g., DNA or RNA) sequence that comprises partial length or entire length coding sequences necessary for the production of a polypeptide or precursor polypeptide.

A “lipid particle” is used herein to refer to a lipid formulation that can be used to deliver an active agent or therapeutic agent, such as a nucleic acid (e.g., an mRNA), to a target site of interest. In the lipid particle of the present disclosure, which is typically formed from a cationic lipid, a non-cationic lipid, and a conjugated lipid that prevents aggregation of the particle, the active agent or therapeutic agent can be encapsulated in the lipid, thereby protecting the agent from enzymatic degradation.

The term “lipid conjugate” refers to a conjugated lipid that inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides, cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In some embodiments, non-ester containing linker moieties are used.

The term “amphipathic lipid” refers, in part, to any suitable material wherein the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “cationic lipid” refers to any of a number of lipid species that carry a net positive charge at a selected pH, such as physiological pH (e.g., pH of about 7.0). Cationic lipids comprising alkyl chains with multiple sites of unsaturation, e.g., at least two or three sites of unsaturation, can be useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 8,058,069; 5,283,185; 5,753,613; and 5,785,992. Non-limiting examples of cationic lipids are described in detail herein. In some cases, the cationic lipids comprise a protonatable tertiary amine (e.g., pH titratable) head group, C18 alkyl chains, ether linkages between the head group and alkyl chains, and 0 to 3 double bonds. Such lipids include, e.g., DSDMA, DLinDMA, DLenDMA, and DODMA.

The term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

Any aspect or embodiment described herein can be combined with any other aspect or embodiment as disclosed herein.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Increasing Lifespan in a Mammal by Overexpressing STC-2 Via Administration of a Single, Nucleic-Acid Containing Composition

In this example, a mammal is administered a single composition comprising a nucleic acid that encodes a fragment of STC-2 or a modified protein which comprises a fragment of STC-2 and a modification that improves the half-life of the fragment of STC-2 in serum. The composition delivers the nucleic acids to cells in the mammal, which synthesize the STC-2 fragment or the modified protein.

Here, a mature mammal, either a mouse, dog, or human, is administered a composition comprising the nucleic acid, which may be a synthetic RNA (e.g., mRNA) encapsulated in a lipid nanoparticle or liposome. In some cases, the nucleic acid is unencapsulated, e.g., comprising naked mRNA. A cell that uptakes the nucleic acid then directly (for mRNA) or indirectly (for DNA) translates the nucleic acid to produce the STC-2 fragment or modified protein. The STC-2 fragment or modified protein is secreted from the cell and binds pregnancy-associated plasma protein-A (PAPP-A) and/or PAPP-A2 and inhibits their enzymatic activity.

Administration is by intravenous injection or infusion, with a dose depending on the quantity of composition needing to be administered.

A first blood sample is collected from the mammal before administering the composition and a second blood sample is collected from the mammal after administering the composition. The blood samples are screened for the presence and amounts of various biomarkers that are relevant to lifespan, aging, and/or healthspan; comparisons are made between results from the first sample and the second sample. In certain cases, only a blood sample is collected from the mammal after administering the composition and comparisons are made between results from the collected sample and historical controls.

The mammal's lifespan is measured and the presence, absence, and/or severity of various aging-associated conditions are determined; these are compared to control mammals and/or to historical controls to determine the effectiveness of the composition administered.

When the mammal has a cancer, administering the composition treats the cancer and/or delays or prevents the onset of the cancer.

### Example 2: Increasing Lifespan in a Mammal by Overexpressing STC-2 Via Administration of Multiple, Nucleic-Acid Containing Compositions

In this example, a mammal is administered multiple compositions each comprising a nucleic acid that encodes a fragment of STC-2 or a modified protein, which comprises a fragment of STC-2 and a modification that improves the half-life of the fragment of STC-2 in serum The compositions deliver the nucleic acids to cells in the mammal, which synthesize the STC-2 fragment or the modified protein.

Here, a mature mammal, either a mouse, dog, or human, is administered a first composition and an, at least, second composition. Each composition comprises a nucleic acid, which may be a synthetic RNA (e.g., mRNA), encapsulated in a lipid nanoparticle or liposome. In some cases, the nucleic acid is unencapsulated, e.g., comprising naked mRNA. A cell that uptakes the nucleic acid then directly (for mRNA) or indirectly (for DNA) translates the nucleic acid to produce the STC-2 fragment or modified protein. The STC-2 fragment or modified protein is secreted from the cell and binds pregnancy-associated plasma protein-A (PAPP-A) and/or PAPP-A2 and inhibits their enzymatic activity.

Administration of the first composition and the, at least, second composition are each by intravenous injection or infusion, with the doses depending on the quantity of compositions needing to be administered. The second composition is administered after a defined period of time after the first composition is administered. The defined period of time depends on the characteristics including species, strain, breed, sex, age, weight, size, health, and/or disease status of the mammal and the particular nucleic acid included in the composition.

A first blood sample is collected from the mammal before administering the first composition, a second blood sample is collected from the mammal after administering the first composition, and a third blood sample is collected from the mammal after administering the second composition. The blood samples are screened for the presence and amounts of various biomarkers that are relevant to lifespan, aging, and/or healthspan; comparisons are made between results from the first sample, the second sample, and the third sample. In certain cases, only a blood sample is collected from the mammal after administering the first composition or only a blood sample is collected from the mammal after administering the second composition and comparisons are made between results from the collected samples. In other cases, only one or more blood samples are collected after administering the first compositions and/or after administering the second composition, but not before administering the first composition; here, comparisons are made between results from the collected sample(s) and historical controls.

The mammal's lifespan is measured and the presence, absence, and/or severity of various aging-associated conditions are determined; these are compared to control mammals and/or to historical controls to determine the effectiveness of the compositions administered.

When the mammal has a cancer, administering the compositions treat the cancer and/or delays or prevents the onset of the cancer.

### Example 3: Increasing Lifespan in a Mammal by Overexpressing STC-2 Via a Single Composition Comprising a Viral Expression Vector

In this example, a mammal is administered composition comprising a viral expression vector including a nucleic acid that encodes a fragment of STC-2 or a modified protein, which comprises a fragment of STC-2 and a modification that improves the half-life of the fragment of STC-2 in serum. The viral expression vectors deliver the nucleic acid to a cell in the mammal, which synthesize the STC-2 fragment or the modified protein.

Here, a mature mammal, either a mouse, dog, or human, is administered a composition comprising adeno-associated virus 2 (AAV2) or AAV8 particles containing a nucleic acid that encodes a fragment of STC-2 or a modified protein, which comprises a fragment of STC-2 and a modification that improves the half-life of the fragment of STC-2 in serum. A cell that is infected with the viral particle expresses the nucleic acid as mRNA which is then translated to produce the STC-2 fragment or modified protein. The STC-2 fragment or modified protein is secreted from the cell and binds pregnancy-associated plasma protein-A (PAPP-A) and/or PAPP-A2 and inhibits their enzymatic activity.

Administration is by intravenous injection or infusion, with a dose depending on the quantity of composition needing to be administered.

A first blood sample is collected from the mammal before administering the composition and a second blood sample is collected from the mammal after administering the composition. The blood samples are screened for the presence and amounts of various biomarkers that are relevant to lifespan, aging, and/or healthspan; comparisons are made between results from the first sample and the second sample. In certain cases, only a blood sample is collected from the mammal after administering the composition and comparisons are made between results from the collected sample and historical controls.

The mammal's lifespan is measured and the presence, absence, and/or severity of various aging-associated conditions are determined; these are compared to control mammals and/or to historical controls to determine the effectiveness of the composition administered.

When the mammal has a cancer, administering the composition treats the cancer and/or delays or prevents the onset of the cancer.

### Example 4: Increasing Lifespan in a Mammal by Overexpressing STC-2 Via Multiple Compositions Each Comprising a Viral Expression Vector

In this example, a mammal is administered multiple compositions each comprising a viral expression vector including a nucleic acid that encodes a fragment of STC-2 or a modified protein, which comprises a fragment of STC-2 and a modification that improves the half-life of the fragment of STC-2 in serum. The viral expression vectors deliver the nucleic acid to cells in the mammal, which synthesize the STC-2 fragment or the modified protein.

Here, a mature mammal, either a mouse, dog, or human, is administered a first composition and an, at least, second composition. Each composition comprising adeno-associated virus 2 (AAV2) or AAV8 particles containing a nucleic acid that encodes a fragment of STC-2 or a modified protein comprising a fragment of STC-2 and a modification that improves the half-life of the fragment of STC-2 in serum. A cell that is infected with the viral particle expresses the nucleic acid as mRNA which is then translated to produce the STC-2 fragment or modified protein. The STC-2 fragment or modified protein is secreted from the cell and binds pregnancy-associated plasma protein-A (PAPP-A) and/or PAPP-A2 and inhibits their enzymatic activity.

Administration of the first composition and the, at least, second composition are each by intravenous injection or infusion, with the doses depending on the quantity of compositions needing to be administered. The second composition is administered after a defined period of time after the first composition is administered. The defined period of time depends on the characteristics including species, strain, breed, sex, age, weight, size, health, and/or disease status of the mammal and the particular nucleic acid and/or viral particle included in the composition.

A first blood sample is collected from the mammal before administering the first composition, a second blood sample is collected from the mammal after administering the first composition, and a third blood sample is collected from the mammal after administering the second composition. The blood samples are screened for the presence and amounts of various biomarkers that are relevant to lifespan, aging, and/or healthspan; comparisons are made between results from the first sample, the second sample, and the third sample. In certain cases, only a blood sample is collected from the mammal after administering the first composition or only a blood sample is collected from the mammal after administering the second composition and comparisons are made between results from the collected samples. In other cases, only one or more blood samples are collected after administering the first compositions and/or after administering the second composition, but not before administering the first composition; here, comparisons are made between results from the collected sample(s) and historical controls.

The mammal's lifespan is measured and the presence, absence, and/or severity of various aging-associated conditions are determined; these are compared to control mammals and/or to historical controls to determine the effectiveness of the compositions administered.

When the mammal has a cancer, administering the composition treats the cancer and/or delays or prevents the onset of the cancer.

### Example 5: Increasing Lifespan in a Mammal by Using a Composition or Compositions Comprising STC-2 Fragment-Containing Proteins

In this example, a mammal is administered one or more compositions each comprising an in vitro translated protein comprising an STC-2 fragment or a modified protein, which comprises an STC-2 fragment and a modification that improves the half-life of the fragment of STC-2 in serum.

Here, a mature mammal, either a mouse, dog, or human, is administered one or more compositions, each comprising an in vitro translated protein comprising an STC-2 fragment or a modified protein, which comprises an STC-2 fragment and a modification that improves the half-life of the fragment of STC-2 in serum. In some cases, the STC-2 fragment or modified protein is encapsulated in a lipid nanoparticle or liposome. The STC-2 fragment or modified protein binds pregnancy-associated plasma protein-A (PAPP-A) and/or PAPP-A2 and inhibits their enzymatic activity.

Administration is by intravenous injection or infusion, with a dose depending on the quantity of composition needing to be administered.

A first blood sample is collected from the mammal before administering the composition and a second blood sample is collected from the mammal after administering the composition. The blood samples are screened for the presence and amounts of various biomarkers that are relevant to lifespan, aging, and/or healthspan; comparisons are made between results from the first sample and the second sample. In certain cases, only a blood sample is collected from the mammal after administering the composition and comparisons are made between results from the collected sample and historical controls.

In some cases, a mammal will be administered more than one composition. A second composition is administered after a defined period of time after the first composition is administered. The defined period of time depends on the characteristics including species, strain, breed, sex, age, weight, size, health, and/or disease status of the mammal and the particular protein included in the composition.

The mammal's lifespan is measured and the presence, absence, and/or severity of various aging-associated conditions are determined; these are compared to control mammals and/or to historical controls to determine the effectiveness of the composition administered.

When the mammal has a cancer, administering the composition treats the cancer and/or delays or prevents the onset of the cancer.

### Example 6: Increasing Lifespan in a Mammal by Using a Composition or Compositions Comprising Peptide Growth Hormone Receptor Antagonists, Growth Hormone Releasing Hormone Antagonists, and Additional Peptide Inhibitors of PAPP-A

This example is conducted similarly as Example 5, except that STC-2 is replaced with a peptide growth hormone receptor antagonist, a growth hormone releasing hormone antagonist, or a different peptide inhibitor of PAPP-A.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed. It is intended that the following claims define the scope of the disclosure and that methods and structures within the scope of these claims and their equivalents be covered thereby.

1. A method for increasing lifespan in a non-rodent mammal in need thereof comprising: administering to the mammal a therapeutically effective amount of a composition comprising a lipid nanoparticle or liposome, wherein the lipid nanoparticle or liposome comprises a nucleic acid encoding a target protein or a functional fragment or variant thereof.

2. (canceled)

3. The method of claim 1, wherein the lipid nanoparticle or liposome comprises at least one of the following: (a) a cationic lipid; (b) a phospholipid; (c) a cholesterol or a derivative thereof; and/or (d) a conjugated lipid that inhibits aggregation of particles.

4. The method of claim 3, wherein the nucleic acid comprises RNA.

5. The method of claim 4, wherein the RNA is an mRNA comprising one or more of cap structure, a Kozak consensus sequence, a 5′-UTR, a 3′-UTR, and a 3′ poly(A) tail.

6. The method of claim 3, wherein the nucleic acid comprises DNA that is single stranded or double stranded.

7. The method of claim 6, wherein the DNA is linear DNA or circular DNA.

8. (canceled)

9. (canceled)

10. The method of claim 7, wherein the DNA comprises a termination and/or polyadenylation sequence.

11. The method of claim 10, wherein the DNA comprises an SV40, hGH, BGH, or rbGlob termination and/or polyadenylation sequence.

12. The method of claim 1, wherein the administering is repeated at least one additional time.

13.-16. (canceled)

17. The method of claim 1, wherein administering the composition comprises intravenous injection or infusion, intraperitoneal injection, intramuscular injection, or subcutaneous injection.

18. The method of claim 1, wherein the nucleic acid encodes a fragment of stanniocalcin-2 (STC-2).

19. A method for increasing lifespan in a non-rodent mammal in need thereof comprising: administering to the mammal a therapeutically effective amount of a composition including a modified protein comprising a target protein or a functional fragment or variant thereof and a modification that improves the half-life of the target protein or the functional fragment or variant thereof in serum.

20. (canceled)

21. The method of claim 19, wherein the modified protein is incapable of crossing the blood brain barrier.

22. (canceled)

23. The method of claim 19, wherein the modification that improves the half-life of the modified protein in serum is derived from an Fc domain of an antibody, a transferrin, an albumin, a CTP sequence, an XTEN sequence, an ELP sequence, a PAS sequence, a HAP sequence, and a GLK sequences, or a combination thereof.

24. The method of claim 23, wherein the antibody is IgG (e.g., IgG1, IgG2, IgG3, and IgG4), IgA (e.g., IgA1 and IgA2), IgD, and IgE.

25. The method of claim 24, wherein the Fc domain is from an IgG4 and comprises one or more mutations selected from S228P, T250Q, M428L, V308T, L309P, and Q311S in accordance with Kabat numbering.

26.-29. (canceled)

30. The method of claim 19, wherein the target protein comprises or is a fragment of stanniocalcin-2 (STC-2), a peptide growth hormone receptor antagonist, or a peptide inhibitor of PAPP-A.

31. The method of claim 30, wherein the fragment of STC-2 is capable of binding pregnancy-associated plasma protein-A (PAPP-A) and/or PAPP-A2 and inhibiting their enzymatic activity.

32.-50. (canceled)

51. The method of claim 1, wherein administering the composition further improves the healthspan of the mammal.

52.-64. (canceled)

65. A method for increasing lifespan in a non-rodent mammal in need thereof comprising: administering to the mammal a therapeutically effective amount of a composition comprising a nucleic acid encoding a target protein or a functional fragment or variant thereof, wherein the composition comprises a viral expression vector.

66.-72. (canceled)

73. A lipid nanoparticle composition for use in a method of increasing lifespan in a non-rodent mammal, wherein the lipid nanoparticle comprises (i) a nucleic acid encoding a target protein or a functional fragment or variant thereof and (ii) one or more of the following: (a) a cationic lipid; (b) a phospholipid; (c) a cholesterol or a derivative thereof; and/or (d) a conjugated lipid that inhibits aggregation of particles.

74.-75. (canceled)
